(19) United States
(12) Patent Application Publication
(10) Pub. No.: US 2024/0382606 A1
(43) Pub. Date: Nov. 21, 2024

(54) NANOPARTICLES FOR ANTIGEN-SPECIFIC CELL PROGRAMMING AND USES THEREOF

(71) Applicant: Georgia Tech Research Corporation, Atlanta, GA (US)

(72) Inventors: Gabriel A. Kwong, Atlanta, GA (US); Shreyas Dahotre, Atlanta, GA (US); Marielena Gamboa, Atlanta, GA (US); Philip J. Santangelo, Atlanta, GA (US); Fang-Yi Su, Atlanta, GA (US); Daryll Vanover, Atlanta, GA (US)

(21) Appl. No.: 18/287,588

(22) PCT Filed: Apr. 20, 2022
(86) PCT No.: PCT/US2022/025645

§ 371 Date: Oct. 19, 2023

Related U.S. Application Data
(60) Provisional application No. 63/176,970, filed on Apr. 20, 2021.
(60) Provisional application No. 63/312,483, filed on Feb. 22, 2022.

(51) Int. Cl. A61K 47/64; A61K 39/00; A61K 39/12; A61K 39/145; A61K 39/21; A61K 39/215; A61K 39/245; A61K 47/54; A61K 47/69

(52) U.S. Cl. CPC A61K 47/646; A61K 39/001102; A61K 39/12; A61K 39/145; A61K 39/21; A61K 39/215; A61K 39/245; A61K 47/549; A61K 47/6909; A61K 2039/53; A61K 2039/605

(57) ABSTRACT

The present disclosure relates to nanoparticle compositions for modifying antigen-specific T cells and uses thereof.

### CROSS-REFERENCE TO RELATED APPLICATIONS

This application claims the benefit of U.S. Provisional Application No. 63/176,970, filed on Apr. 20, 2021, and U.S. Provisional Application No. 63/312,483, filed on Feb. 22, 2022, which are expressly incorporated herein by reference in their entirety.

### STATEMENT REGARDING FEDERALLY SPONSORED RESEARCH

This invention was made with government support under grant no. GR10025308 awarded by the Defense Advanced Research Projects Agency (DARPA). The government has certain rights in the invention.

### FIELD

The present disclosure relates to compositions for antigen-specific cell programming and uses thereof.

### BACKGROUND

T cell-based therapies have risen to the forefront of biomedicine, providing unprecedented opportunities for the successful treatment of a broad spectrum of diseases. Remarkably, T cells can recognize unique fragments of antigens as peptides presented by major histocompatibility complex (MHC) with exquisite sensitivity to clear diseases and generate long-term immune protection. Following viral infections, for instance, virus-specific CD8 T cells are generated with memory phenotype that persist for several years to generate protection against subsequent viral infections. Conversely, dysregulation of antigen-specific T cell populations can promote disease progression. For instance, many cancers leverage immune checkpoints (e.g., PD-1, CTLA-4) to suppress and interfere with T cells specific for tumor associated antigens (TAAs). T cells can also become hypersensitive to self-antigens in the case of autoimmune diseases (e.g., multiple sclerosis, type-1 diabetes) or lose tolerance to an otherwise innocuous antigen in allergy. Current therapies focus on systemic modulation of T cell activity, yet global inhibition of all T cell clones may leave patients immunocompromised and vulnerable to opportunistic infections, while global activation can lead to hyperactive T cells that can lead to severe off-target toxicity.

There is a need for tight control over the production and activity of antigen-specific T cells. There are currently two major approaches to generate or modulate antigen-specific T cells. The first approach relies on ex vivo T cell isolation and use viral vector to genetically engineer T cells to express artificial T cell receptors (e.g., chimeric antigen receptors, CAR) that target specific antigens (e.g., tumor antigens). While this approach has been proved by FDA for the treatment of B cell malignancies, its broad clinical application is limited by lengthy (3-5+ weeks) and costly ($350K-450K) manufacturing processes. The second approach is using vaccines to stimulate and expand T cells in vivo in an antigen-specific manner. While vaccines have been broadly used for disease prevention (e.g., smallpox, yellow fever), vaccines do not always induce effective T cell responses (e.g., inactivated influenza vaccines), nor provide the flexibility to program selective genes in antigen-specific T cells (e.g., endow T cells with customizable antigen-specificity, induce sustained T cell immunity by suppressing checkpoint inhibitor PD-1). Therefore, what is needed are new compositions and methods for programming antigen-specific cells.

### SUMMARY

In some aspects, disclosed herein is a composition comprising:
a nanoparticle;
a major histocompatibility complex (MHC) molecule; and
a peptide.

In some embodiments, the MHC molecule comprises an MHC class 1 molecule or an MHC class 2 II molecule. In some embodiments, the MHC molecule comprises HLA-A*01:01, HLA-A*02:01, HLA-A*03:01, HLA-A*07:02, HLA-A*11:01, or HLA-A*24:02. In some embodiments, the MHC class I molecule comprises a heavy chain that comprises a C-terminal cysteine. In some embodiments, the heavy chain comprises a sequence at least 60% identical to one of SEQ ID NOS: 23-25.

In some embodiments, the peptide binds to the MHC molecule. In some embodiments, the peptide is a peptide fragment of a human protein or a non-human protein. In some embodiments, the non-human protein is a viral protein. In some embodiments, the viral protein is an influenza virus protein, a human papillomavirus protein, a human immunodeficiency virus protein, a lymphocytic choriomeningitis virus protein, a cytomegalovirus protein, an Epstein-Barr virus protein, or a SARS-CoV-2 protein.

In some embodiments, the human protein is expressed on a cancer cell.

In some embodiments, the peptide comprises a sequence at least 80% identical to one of SEQ ID NOs: 1-9.

In some embodiments, the nanoparticle is a lipid nanoparticle. In some embodiments, the lipid nanoparticle comprises MC3, phosphatidylcholine (1,2-distearoyl-sn-glycero-3-phosphocholin (DSPC), cholesterol, distearoyl glycerol-polyethylene glycol (DMG-PEG), or 1,2-Distearoyl-sn-glycero-3-phosphorylethanolamine (DSPE)-PEG.

The MHC molecule and the peptide can be located on the surface of the nanoparticle. In some embodiments, the MHC molecule is conjugated to a linker at the C-terminus. In some embodiments, the MHC molecule is linked to the nanoparticle through the linker. In some embodiments, the linker comprises 1,2-Distearoyl-sn-glycero-3-phosphorylethanolamine (DSPE), polyethylene glycol (PEG), or maleimide.

In some embodiments, the nanoparticle further comprises a nucleic acid. In some embodiments, the nucleic acid encodes a chimeric antigen receptor, a CRISPR/Cas9 endonuclease, a costimulatory molecule, and/or a cytokine. In some embodiments, the nucleic acid is RNA or DNA.

Disclosed herein is a method of treating a cancer in a subject in need, comprising administering to the subject a therapeutically effective amount of the composition of any preceding aspect.

Also disclosed herein is a method of treating an autoimmune disorder in a subject in need, comprising administering to the subject a therapeutically effective amount of the composition of any preceding aspect.

Further disclosed herein is a high throughput method of creating a composition that comprises a nanoparticle, a major histocompatibility complex (MHC) molecule, and a peptide, said method comprising:
attaching the MHC molecule to a sacrificial peptide;
contacting the MHC molecule with a lipid micelle to create a first MHC micelle;
contacting the first MHC micelle with the peptide;
applying a UV light to the first MHC micelle and the peptide to create a second MHC micelle; and
creating the composition by contacting the second MHC micelle with the nanoparticle.

### BRIEF DESCRIPTION OF DRAWINGS

The accompanying figures, which are incorporated in and constitute a part of this specification, illustrate several aspects described below.

FIGS. 1A-1B show schematic of UV-mediated peptide exchange of MHCI antigen-presenting nanoparticles (APNs) for in vivo multiplexed delivery to virus-specific T cells. (FIG. 1A) pMHC molecules were refolded with photolabile peptides and then conjugated them to a lipid tail to allow subsequent formulation with preformed lipid nanoparticles (NPs). The presence of UV light cleaves the photolabile peptide and induces replacement of the resulting empty MHC groove with a library of viral peptides. After the UV-mediated peptide ligand exchange, pMHC molecules were functionalize on the surface of preformed NPs via postinsertion to form the APN library for multiplexed delivery to virus-specific T cells. (FIG. 1B) After intravenous injection into living mice, APNs selectively target cognate T cell populations and transfect them with model mRNA. The mRNA expression was validate using flow analysis.

FIGS. 2A-2F show that APNs target Ag-specific T cells and induce cell uptake in vitro. (FIG. 2A) Illustration of the interaction of P14 CD8+ T cells with its cognate APNs (GP33/Db), in contrast to the lack of binding to the noncognate control (GP100/Db APN). (FIG. 2B) Representative flow plots of noncognate APNs and cognate APNs binding to CD8+ T cells in splenocytes from P14 TCR transgenic mice. Frequencies depicted are based on gating on CD8+ cells. (FIG. 2C) Ag-specific binding of cognate and noncognate APNs to CD8+ T cells isolated from TCR transgenic P14 or OT-1 mice. ****P<0.0001, one-way analysis of variance (ANOVA) and Tukey post-test and correction. All data are means±SD; n=3 biologically independent wells. (FIG. 2D and FIG. 2E) OT-1 CD8+ splenocytes stained with OVA/Kb APNs at 4° or 37° C. and analyzed by flow cytometry before and after treatment with an acetate buffer to strip cell surface proteins. GP100/Db APNs served as a noncognate control. ****P<0.0001 between OVA/Kb APN treatment with and without acid treatment at 4° C.; n.s., not significant, where P=0.61 between OVA/Kb APN with and without acid treatment at 37° C.; two-way ANOVA and Sidak post-test and correction. All data are means±SD; n=3 biologically independent wells. (FIG. 2F) eGFP mRNA expression in P14 CD8+ T cells after coincubation with free-form eGFP mRNA or eGFP mRNA loaded in GP33/Db APNs for 24 hours.

FIGS. 3A-3F show that APNs target and transfect Ag-specific T cells in TCR transgenic P14 mice. (FIG. 3A) Intravenous injection of GP33/Db APNs to TCR transgenic P14 mice. mRNA encoding Fluc or camelid antibody VHH was loaded in APNs as a reporter. Major organs or splenocytes were harvested for IVIS imaging of Fluc expression or flow analysis of VHH protein expression on the CD8+ splenocytes. GP100/Db APNs were used as a noncognate control. (FIG. 3B) Representative bioluminescence images of various organs were recorded after 6 hours after APN injection to P14 mice. (FIG. 3C) Quantification data of bioluminescence images show in (FIG. 3B). (FIG. 3D) In vivo targeting of GP33/Db APNs to CD8+ splenocytes in P14 mice. *P=0.0132 between PBS and GP100/Db APNs; ****P<0.0001 between PBS and GP33/Db APNs and between GP100/Db APNs and GP33/Db APNs; one-way ANOVA and Tukey post-test and correction. (FIG. 3E) Representative flow plot showing in vivo APN-mediated transfection in P14 CD8+ splenocytes. (FIG. 3F) In vivo APN-mediated transfection in both P14 CD8− and CD8+ splenocytes. ****P<0.0001 between PBS and GP33/Db APNs and between GP100/Db APNs and GP33/Db APNs; one-way ANOVA and Tukey post-test and correction. All data are means±SD; n=5 biologically independent mice.

FIGS. 4A-4F show UV-exchanged APNs transfect Ag-specific T cells equivalently to folded APNs. (FIG. 4A) Light-triggered peptide exchange technology for high-throughput production of pMHC molecules with various peptide epitopes. pMHC molecules were folded with photolabile peptides that can be cleaved and exchanged with target peptides, followed by tetramer formation with streptavidin conjugated with PE. (FIG. 4B) Using the UV-exchanged tetramer library to stain virus-specific T cells in a mouse model of PR8-GP33 flu infection. (FIG. 4C) Flow cytometry validation of five epitopes showing diverse Ag specificity of T cell responses to PR8-GP33 flu infection. (FIG. 4D) Equivalent CD8⁺ splenocyte binding efficiency of UV-exchanged APNs to conventionally folded APNs in three TCR transgenic mouse models in vitro. Numbers indicate the percentage of APN⁺ cells of CD8⁺ cells. (FIG. 4E) In vivo targeting activity of UV-exchanged PA224/D^{b} APNs and folded PA224/D^{b} APNs to PA224-specific CD8⁺ T cells in a mouse model of PR8 infection. n.s., not significant, where P=0.4548 between folded and UV-exchanged PA224/Db APNs; ****P<0.0001 between PBS and UV-exchanged PA224/Db APNs; one-way analysis of variance (ANOVA) and Tukey post-test and correction. (FIG. 4F) In vivo transfection efficiency of UV-exchanged PA224/D^{b} APNs and folded PA224/D^{b} APNs to PA224-specific CD8⁺ T cells in a mouse model of PR8 infection. n.s., not significant, where P=0.5191 between folded and UV-exchanged PA224/Db APNs; ****P<0.0001 between PBS and UV-exchanged PA224/D^{b} APNs; one-way ANOVA and Tukey post-test and correction. For (FIG. 4E) and (FIG. 4F), all data are means±SD; n=6 biologically independent mice.

FIGS. 5A-5E show that APNs transfect multiplexed T cell subsets with significantly higher transfection efficiency than noncognate cell populations. (FIG. 5A) Schematic of functional biodistribution study at cellular level comparing UV-exchanged PA224/D^{b} APNs with folded PA224/D^{b} APNs. (FIG. 5B) Transfection efficiency of PBS and PA224/D^{b} APNs in the major cell populations of spleen and liver. **P=0.0010 between PBS and UV-exchanged PA224/D^{b} APNs in monocyte cell population; **P=0.0025 between PBS and UV-exchanged PA224/D^{b} APNs in Kupffer cells; ****P<0.0001; two-way ANOVA and Dunnett post-test and correction. All data are means±SD; n=4 to 5 biologically independent mice. (FIG. 5C) Dose-dependent transfection in two immunodominant flu-specific T cells in PR8 model. Infected mice were treated with a mixture of folded NP366/D^{b} APNs and PA224/D^{b} APNs. n.s., not significant, where P=0.0606 between PBS and folded APNs (0.03 mg/kg total mRNA dose) in NP366+ flu-specific T cell population; **P=0.0015 between PBS and folded APNs (0.03 mg/kg) in PA224+ flu-specific T cell population (PA224+); ****P<0.0001; two-way ANOVA and Sidak post-test and correction. All data are means±SD; n=3 biologically independent mice. (FIG. 5D) Schematic of multiplexed transfection study. (FIG. 5E) Multiplexed transfection study showing the UV-exchanged APN library transfect three virus-specific T cell populations simultaneously. ****P<0.0001; two-way ANOVA and Sidak post-test and correction. All data are means±SD; n=5 biologically independent mice.

FIGS. 6A-6C. Synthesis and characterization of antigen-presenting nanoparticles (APNs). (FIG. 6A) Schematic of the generation of APNs using microfluidic mixers and post-insertion technique. (FIG. 6B) Uniform distribution of the synthesized bare nanoparticles (NPs) and APNs functionalized with GP33/D^{b} APN. (FIG. 6C) Hydrodynamic size, polydispersity index (PDI), zeta potential, and mRNA loading of the produced NPs. Data are presented as mean±S.D. n=3 technical replicates.

FIG. 7. In vivo APN-mediated transfection in CD8+ splenocytes isolated from Pmel TCR transgenic mice. ***P<0.001 between GP33/D^{b} APNs (non-cognate) and GP100/D^{b} APNs (cognate); one-way ANOVA and Tukey post-test and correction. All data are means±SD; n=3 biologically independent mice.

FIG. 8. UV tetramers (unexchanged) did not cause non-non-specific binding to CD8+ splenocytes from P14 and Pmel TCR transgenic mice. Representative flow plots of CD8+ splenocytes staining with UV tetramers or UV exchanged cognate tetramers. Frequencies depicted are based on gating on CD8+ cells.

FIG. 9. UV-exchanged tetramers bound to P14 CD8 splenocytes comparably to the folded counterpart. Dot plots of flow cytometry analysis for P14 splenocytes stained with cognate (GP33/D^{b}) tetramer and non-cognate (GP100/D^{b}) tetramer prepared by conventional folding protocol (folded) or UV-mediated ligand exchange protocol (UV).

FIG. 10. Biodistribution of DiR-labeled APNs. Quantification of DiR fluorescent signal in each organ. Each symbol indicates one measured organ. All data are means±SD; n=3 biologically independent mice.

FIG. 11. Dose-dependent APN targeting to two immunodominant flu-specific T cells in PR8 model. Infected mice were intravenously injected with a mixture of folded NP366/D^{b} APNs and PA224/D^{b} APNs. n.s.=not significant where P>0.9999 between PBS and folded APNs (0.03 mg/kg total mRNA dose) as well as between PBS and folded APNs (0.2 mg/kg) in NP366-PA224-T cells; **P=0.0033 between PBS and folded APNs (0.03 mg/kg) in NP366+ flu-specific T cell population (NP366+); ****P<0.0001; two-way ANOVA and Sidak post-test and correction. All data are means±SD; n=3 biologically independent mice.

FIGS. 12A-12B. pMHC-LNPs target mouse CD8+ T cells ex vivo in an antigen-specific manner. (FIG. 12a) Schematic of the targeting specificity of pMHC-LNPs. (FIG. 12b) LNPs functionalized with gp33 pMHC interacted with T cells harvested from p14 transgenic mice (matched TCR), but not T cells harvest from pmel transgenic mice (mismatched TCR). TCR: T cell receptor.

FIG. 13. pMHC LNPs deliver cargo intracellularly, not the surface of the cell. Representative histograms for p14 CD8+ T cells stained with pMHC-LNPs at 37° C. and analyzed by flow cytometry before and after treatment with an acid wash to strip cell surface receptors.

FIGS. 14A-14B. pMHC-LNPs specifically target TCR-matched human CD8+ T cells ex vivo. (FIG. 14a) Schematic showing the workflow of pMHC-LNPs targeting to human CMV-specific T cells isolated from human blood. (FIG. 14b) Flow plot demonstrating that pMHC-LNPs specifically target to CMV-specific T cells, but not other CD8+ T cells present in the human PBMC.

FIG. 15. Method for UV-mediated ligand exchange of pMHC to control targeting specificity of pMHC-LNPs.

FIG. 16. UV-exchanged pMHC-LNPs show comparable size distribution and T cell targeting as wild type (WT) pMHC-LNPs.

FIGS. 17A-17D. UV exchanged pMHC-LNPs bind to target CD8+ T cells similarly to gold standard tetramers. (FIG. 17a) Untreated splenocytes harvested from p14 transgenic mice. (FIG. 17b) p14 splenocytes treated with a gold standard gp33 tetramers. (FIG. 17c) p14 splenocytes treated with pMHC-LNP carrying mismatched UV peptide ligands (unexchanged). (FIG. 17d) p14 splenocytes treated with pMHC-LNP carrying matched gp33 peptide ligands (exchanged).

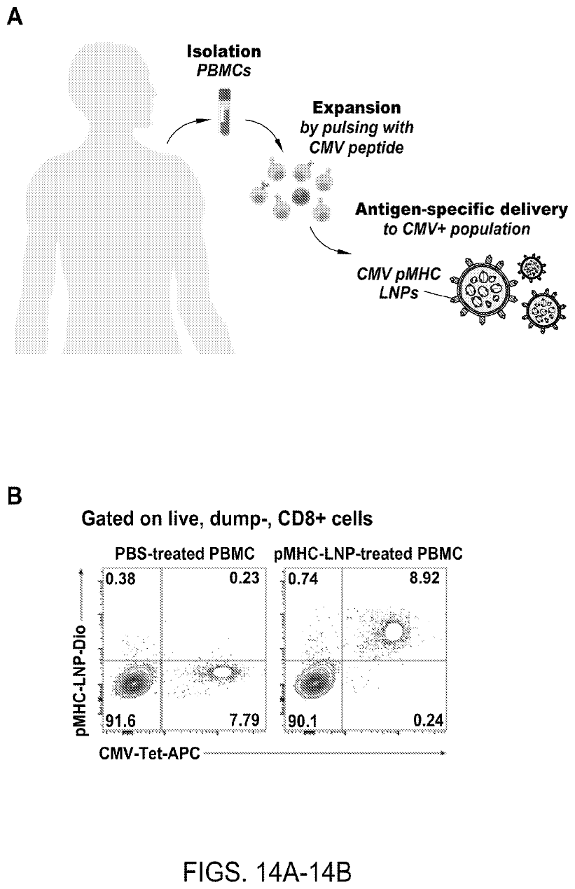

FIG. 18. In vitro eGFP mRNA delivery. Free-form mRNA is unable to transfect T cells. In contrast, pMHC-LNPs effectively transfected p14 CD8+ T cells with a model eGFP mRNA.

FIGS. 19A-19B. In vivo antigen-specific T cell targeting and transfection in flu model. (FIG. 19a) Schematic of workflow to test pMHC-LNP in mice infected with human PR8 flu virus (H1N1). (FIG. 19b) Body weight at 24 hours after LNP treatment.

FIGS. 20A-20D. In vivo T cell transfection with human CD19 mRNA. (FIG. 20a) Schematic showing the workflow of intravenous injection of pMHC-LNPs to p14 transgenic mice to test in vivo transfection efficiency. (FIG. 20b) LNPs were modified with gp33 MHC that can target p14 CD8+ T cells or with mismatched gp100 MHC as a non-targeting control. (FIG. 20c) Bar graph showing the percentage of CD8+ T cells that express CAR on the surface. (FIG. 20d) Representative flow plots of CAR expression on CD8+ T cells in the three treatment groups.

FIGS. 21A-21B. In vivo T cell transfection with mouse CD19 mRNA. (FIG. 21a) Representative flow plots showing pMHCLNPs targeting and transfection p14 CD8+ T cells with mouse CD19 CAR in vivo. (FIG. 21b) Bar graph showing the in vivo transfection efficiency with four biological replicates.

FIGS. 22A-22D. Effector functions of mouse CD19 CAR T cells produced by pMHC-LNPs in vivo. (FIG. 22a) Schematic showing antigen-specific T cell killing of in vivo transfected CAR T cells. (FIG. 22b) Killing of wild type EL4 tumor cells (without mouse CD19 antigen) by T cells. T cells were in vivo transfected by pMHC-LNPs with either targeted or non-targeted MHC. The EL4 cancer cells were co-incubated with T cells for 20 hrs at indicated cell ratios. (FIG. 22c) Killing of mouse CD19-expressing EL4 cells by T cells at the same condition as in FIG. 21a. (FIG. 22d) Interferon gamma (IFN-γ) secretion following the same 20-hr coculture.

FIGS. 23A-23B show that APNs transfect Ag-specific T cells with mRNA encoding endonuclease-dead Cas9 (dCas9) in TCR transgenic OT1 mice. (FIG. 23A) In vivo targeting of OVA/Kb APNs to CD8+ splenocytes in OT1 mice. APNs were loaded with mRNA encoding a fusion protein of dCas9-VPR and VHH (dCas9-VPR-VHH). mRNA transfection was evaluated by VHH staining for flow analysis. APNs loaded with mRNA encoding a fusion protein of dCas9-VPR and nano-Luciferase (dCas9-VPR-nLuc) was used as a negative control for transfection. ***P<0.0001 between PBS and APNs loaded with dCas9-VPR-VHH. One-way ANOVA and Tukey post-test and correction. All data are means±SD; n=3 biologically independent mice. (FIG. 23B) In vivo OVA/Kb APN-mediated transfection in OT1 CD8+ splenocytes. ***P=0.0005 between APNs loaded with dCas9-VPR-nLuc and APNs loaded with dCas9-VPR-VHH. ****P<0.0001 between PBS and APNs loaded with dCas9-VPR-VHH. One-way ANOVA and Tukey post-test and correction. All data are means±SD; n=3 biologically independent mice.

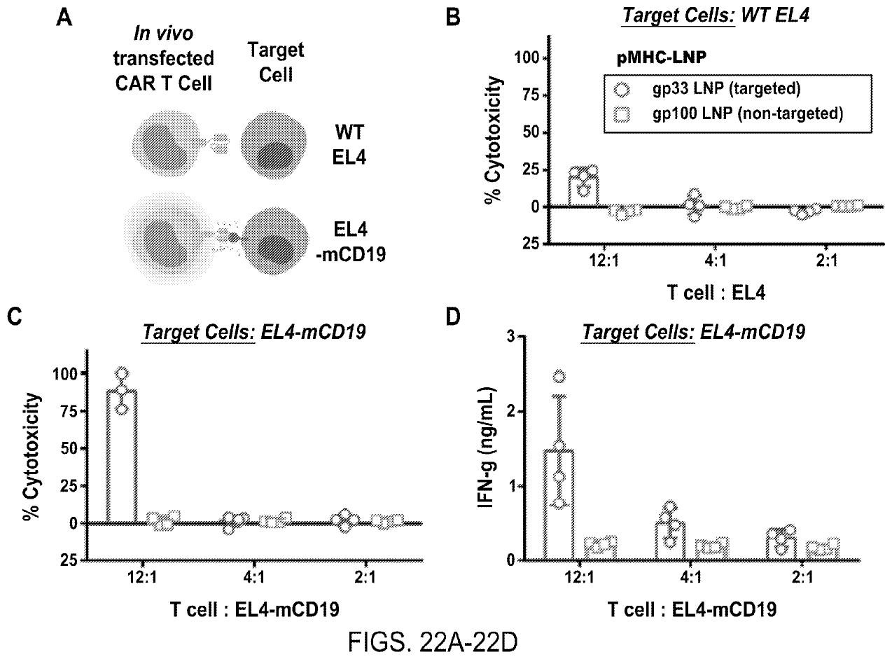

FIG. 24 shows single guide RNA (sgRNA) screening for transcriptional upregulation of mouse IL15 in primary mouse CD8 T cells. Activated CD8 T cells were electroporated with mRNA encoding dCas9-VPR transcriptional activator and sgRNA designed to target the IL15 genome sequence at various locations. 48 hours after electroporation, total mRNA was extracted from T cells for qPCR analysis to evaluate the upregulation of endogenous IL15 at mRNA level. Scrambled sgRNA was included as a negative control. ****P<0.0001 between scrambled sgRNA and IL15 sgRNA #4. One-way ANOVA and Dunnett post-test and correction. All data are means±SD; n=3 biologically intendent wells.

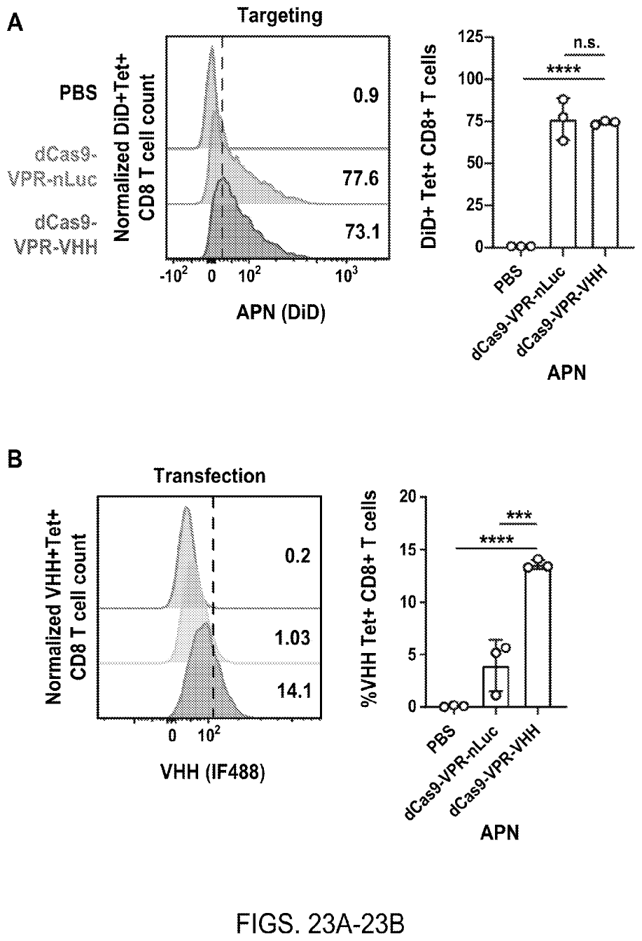

FIG. 25 shows single guide RNA (sgRNA) screening for transcriptional upregulation of mouse IL2 in primary mouse CD8 T cells. Activated CD8 T cells were electroporated with mRNA encoding dCas9-VPR transcriptional activator and sgRNA designed to target the IL2 genome sequence at various locations. 48 hours after electroporation, total mRNA was extracted from T cells for qPCR analysis to evaluate the upregulation of endogenous IL2 at mRNA level. Scrambled sgRNA was included as a negative control. **P=0.0026 between scrambled sgRNA and IL2 sgRNA #1. One-way ANOVA and Dunnett post-test and correction. All data are means±SD; n=3 biologically intendent wells. RNA sequence of IL15 single guide RNA #4: CCUGCUGCAGAGUCUGGAAGG (SEQ ID NO: 29), RNA sequence of IL2 single guide RNA #1: GGUAAUGCUUUCUGCCACAC (SEQ ID NO: 30).

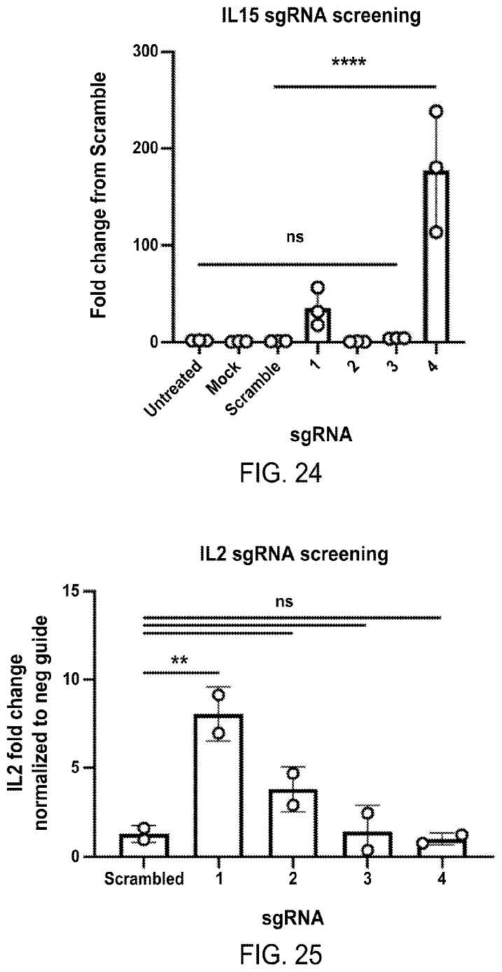

### DETAILED DESCRIPTION

In some aspects, disclosed herein are compositions comprising nanoparticles, major histocompatibility complex (MHC) molecule, and peptides and uses thereof for delivering a therapeutic agent to an antigen-specific cell (e.g., T cell), modifying an antigen-specific cell (e.g., T cell), and treating a disease or disorder. Also disclosed herein is a high throughput method of creating a composition that comprises a nanoparticle, a major histocompatibility complex (MHC) molecule, and a peptide.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

### Terminology

The term “about” as used herein when referring to a measurable value such as an amount, a percentage, and the like, is meant to encompass variations of 20%, +10%, ±5%, or 10% from the measurable value.

“Administration” to a subject or “administering” includes any route of introducing or delivering to a subject an agent. Administration can be carried out by any suitable route, including oral, intravenous, intraperitoneal, intranasal, inhalation and the like. Administration includes self-administration and the administration by another.

As used herein, the terms “antigen” or “immunogen” are used interchangeably to refer to a substance, typically a protein, a nucleic acid, a polysaccharide, a toxin, or a lipid, which is capable of inducing an immune response in a subject. The term also refers to proteins that are immunologically active in the sense that once administered to a subject (either directly or by administering to the subject a nucleotide sequence or vector that encodes the protein) is able to evoke an immune response of the humoral and/or cellular type directed against that protein.

The terms “antigenic determinant” and “epitope” may also be used interchangeably herein, referring to the location on the antigen or target recognized by the antigen-binding molecule (such as the nanobodies of the invention). Epitopes can be formed both from contiguous amino acids (a “linear epitope”) or noncontiguous amino acids juxtaposed by tertiary folding of a protein. The latter epitope, one created by at least some noncontiguous amino acids, is described herein as a “conformational epitope.” An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

The term “autoimmune disease” as used herein is defined as a disorder that results from an autoimmune response. An autoimmune disease is the result of an inappropriate and excessive response to a self-antigen. Examples of autoimmune diseases include but are not limited to, Addison's disease, alopecia greata, ankylosing spondylitis, autoimmune hepatitis, autoimmune parotitis, autoimmune uveitis, Crohn's disease, diabetes (Type 1), celiac disease, dystrophic epidermolysis bullosa, epididymitis, glomerulonephritis, Graves' disease, Goodpasture syndrome, Guillain-Barr syndrome, Hashimoto's disease, hemolytic anemia, inflammatory bowel disease, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, celiac disease, and systemic lupus erythematosus.

The term “biocompatible” generally refers to a material and any metabolites or degradation products thereof that are generally non-toxic to the recipient and do not cause significant adverse effects to the subject.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, multiple myeloma, lymphoma, leukemia, lung cancer and the like.

The term “cancerous cell”, as used herein, generally refers to any cells that exhibit, or are predisposed to exhibiting, unregulated growth. The term “cancer cells” and “tumor cells” are used interchangeably to refer to cells derived from a cancer or a tumor, or from a tumor cell line or a tumor cell culture.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

“Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T/U, or C and G. Two single-stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, at least about 75%, or at least about 90% complementary. See Kanehisa (1984) Nucl. Acids Res. 12:203.

A “control” is an alternative subject or sample used in an experiment for comparison purposes. A control can be “positive” or “negative.”

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount.” However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom, Thus, a gene encodes a protein if transcription and translation of mRNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.)

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the fragment must possess a bioactive property, such as regulating the transcription of the target gene.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

The term “increased” or “increase” as used herein generally means an increase by a statically significant amount; for example, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

An “immunological response” or “immunity” to a composition or vaccine is the development in the host of a cellular and/or antibody-mediated immune response to a composition or vaccine of interest. Usually, an “immunity” includes but is not limited to one or more of the following effects: the production of antibodies, B cells, helper T cells, and/or cytotoxic T cells, directed specifically to an antigen or antigens included in the composition or vaccine of interest. Preferably, the host will display either a therapeutic or protective immunity such that resistance to new infection will be enhanced and/or the clinical severity of the disease reduced.

As used herein, the term “level” refers to the amount of a target molecule in a sample, e.g., a sample from a subject. The amount of the molecule can be determined by any method known in the art and will depend in part on the nature of the molecule (i.e., gene, mRNA, cDNA, protein, enzyme, etc.). The art is familiar with quantification methods for nucleotides (e.g., genes, cDNA, mRNA, etc.) as well as proteins, polypeptides, enzymes, etc. It is understood that the amount or level of a molecule in a sample need not be determined in absolute terms, but can be determined in relative terms (e.g., when compared to a control or a sham or an untreated sample).

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used herein, the term “metastasis” is meant to refer to the process in which cancer cells originating in one organ or part of the body, with or without transit by a body fluid, and relocate to another part of the body and continue to replicate. Metastasized cells can subsequently form tumors which may further metastasize. Metastasis thus refers to the spread of cancer, from the part of the body where it originally occurred, to other parts of the body.

The term “nanoparticle” as used herein refers to a particle or structure which is biocompatible with and sufficiently resistant to chemical and/or physical destruction by the environment of such use so that a sufficient number of the nanoparticles remain substantially intact after delivery to the site of application or treatment and whose size is in the nanometer range. For the purposes of the present invention, a nanoparticle typically ranges from about 1 nm to about 1000 nm, preferably from about 50 nm to about 500 nm, more preferably from about 50 nm to about 350 nm.

The term “nucleic acid” as used herein means a polymer composed of nucleotides, e.g., deoxyribonucleotides (DNA) or ribonucleotides (RNA). The terms “ribonucleic acid” and “RNA” as used herein mean a polymer composed of ribonucleotides. The terms “deoxyribonucleic acid” and “DNA” as used herein mean a polymer composed of deoxyribonucleotides. (Used together with “polynucleotide” and “polypeptide”.)

The term “polypeptide” refers to a compound made up of a single chain of D- or L-amino acids or a mixture of D- and L-amino acids joined by peptide bonds.

The term “polynucleotide” refers to a single or double stranded polymer composed of nucleotide monomers.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ).

The term “primary tumor” refers to a tumor growing at the site of the cancer origin.

The term “polymer” as used herein refers to a relatively high molecular weight organic compound, natural or synthetic, whose structure can be represented by a repeated small unit, the monomer. Synthetic polymers are typically formed by addition or condensation polymerization of monomers. The polymer is suitable for use in the body of a subject, i.e. is biologically inert and physiologically acceptable, non-toxic, and is biodegradable in the environment of use, i.e. can be resorbed by the body. The term “polymer” encompasses all forms of polymers including, but not limited to, natural polymers, synthetic polymers, homopolymers, heteropolymers or copolymers, addition polymers, etc.

The term “copolymer” as used herein refers to a polymer formed from two or more different repeating units (monomer residues). Copolymer compasses all forms copolymers including, but not limited to block polymers, random copolymers, alternating copolymers, or graft copolymers. A “block copolymer” is a polymer formed from multiple sequences or blocks of the same monomer alternating in series with different monomer blocks. Block copolymers are classified according to the number of blocks they contain and how the blocks are arranged.

As used herein, the term “subject” or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human.

The terms “peptide,” “protein,” and “polypeptide” are used interchangeably to refer to a natural or synthetic molecule comprising two or more amino acids linked by the carboxyl group of one amino acid to the alpha amino group of another.

“Recombinant” used in reference to a gene refers herein to a sequence of nucleic acids that are not naturally occurring in the genome of the bacterium. The non-naturally occurring sequence may include a recombination, substitution, deletion, or addition of one or more bases with respect to the nucleic acid sequence originally present in the natural genome of the bacterium.

As used herein, a “target”, “target molecule”, or “target cell” refers to a biomolecule or a cell that can be the focus of a therapeutic drug strategy, diagnostic assay, or a combination thereof, sometimes referred to as a theranostic. Therefore, a target can include, without limitation, many organic molecules that can be produced by a living organism or synthesized, for example, a protein or portion thereof, a peptide, a polysaccharide, an oligosaccharide, a sugar, a glycoprotein, a lipid, a phospholipid, a polynucleotide or portion thereof, an oligonucleotide, an aptamer, a nucleotide, a nucleoside, DNA, RNA, a DNA/RNA chimera, an antibody or fragment thereof, a receptor or a fragment thereof, a receptor ligand, a nucleic acid-protein fusion, a hapten, a nucleic acid, a virus or a portion thereof, an enzyme, a co-factor, a cytokine, a chemokine, as well as small molecules (e.g., a chemical compound), for example, primary metabolites, secondary metabolites, and other biological or chemical molecules that are capable of activating, inhibiting, or modulating a biochemical pathway or process, and/or any other affinity agent, among others.

Therapeutically effective amount” or “therapeutically effective dose” of a composition (e.g. a composition comprising an agent) refers to an amount that is effective to achieve a desired therapeutic result. In some embodiments, a desired therapeutic result is the control of a disorder or a symptom thereof. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

The terms “treat,” “treating,” “treatment,” and grammatical variations thereof as used herein, include partially or completely delaying, alleviating, mitigating or reducing the intensity of one or more attendant symptoms of cancer or condition and/or alleviating, mitigating or impeding one or more symptoms of cancer. Treatments according to the invention may be applied preventively, prophylactically, palliatively or remedially. Prophylactic treatments are administered to a subject prior to onset (e.g., before obvious signs of cancer), during early onset (e.g., upon initial signs and symptoms of cancer), after an established development of cancer, or during prevention or mitigation of cancer relapse. Prophylactic administration can occur for several minutes to months prior to the manifestation of an infection.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

### Nanoparticles and Uses Thereof

Disclosed herein are compositions targeting antigen-specific cells in vivo or ex vivo. In some aspects, disclosed herein is a composition comprising:
a nanoparticle;
a major histocompatibility complex (MHC) molecule; and
a peptide.

The term “MHC molecule” or “major histocompatibility complex molecule” herein refers to the highly polymorphic glycoproteins encoded by MHC class I and MHC class II genes, which are involved in the presentation of peptide antigens to T cells. The function of MHC molecules is to bind peptide fragments and display them on the cell surface for recognition by the appropriate T cells. There are two classes of MHC molecule—MHC class I molecules and MHC class II molecules. In addition to being bound by the T-cell receptor, the two classes of MHC molecule are differentially recognized by the two co-receptor molecules, CD8 and CD4, which characterize the two major subsets of T cells. CD8 T cells recognize MHC class I:peptide complexes. CD4 T cells recognize MHC class II:peptide complexes.

It should be understood and herein contemplated that an MHC class I molecule comprises an a chain (also herein termed as “heavy chain”) that is polymorphic and a beta2-microglobulin that is invariant. An MHC class I molecule comprises a peptide binding groove that binds peptides (also known as “epitopes”). An MHC class II molecule comprises an α and a β chain. The N-terminal domains of each of the α and β chain are polymorphic and important in antigen presentation. In the case of MHC class II, the peptide-binding groove is formed by the interaction of the N-terminal domains of the α and β chains.

In some embodiments, the nanoparticle disclosed herein comprises one or more MHC class I molecules or one or more MHC class II molecules. In some embodiments, the nanoparticle disclosed herein comprises one or more MHC class I molecules and one or more MHC class II molecules. In some embodiments, the nanoparticle disclosed herein comprises one or more MHC class I molecules, wherein the one or more MHC class I molecules are same MHC allele. In some embodiments, the nanoparticle disclosed herein comprise one or more MHC class I molecules, wherein the one or more MHC class I molecules are different MHC alleles.

In some examples, the MHC molecule disclosed herein is a human MHC molecule. The human MHC molecule is also called the “HLA” (human leukocyte antigen). In some embodiments, the composition disclosed herein comprises an MHC class I molecule or an MHC class II molecule. In some embodiments, the human MHC molecule is HLA-A*01:01, HLA-A*02:01, HLA-A*03:01, HLA-A*07:02, HLA-A*11:01, or HLA-A*24:02. In some embodiments, the human MHC molecule is associated with autoimmune diseases, wherein the human MHC molecule is that in Table 2. In some embodiments, the MHC molecule disclosed herein is a mouse MHC molecule. In some embodiments, the mouse MHC molecule is H2-D^{b}, H2-K^{b}, or H2-L^{b}. In some examples, the heavy chain of the MHC class I molecule can comprises a C-terminal cysteine. In some embodiments, the heavy chain comprises a sequence at least 60% identical to SEQ ID NOS: 23-25 or a fragment thereof.

In some embodiments, the peptide binds to the MHC class I molecule. In some embodiments, the peptide binds to the MHC class II molecules.

The peptide can be about 5 to about 50 amino acid residues in length (about 5 to about 30 amino acid residues in length, about 5 to about 20 amino acid residues in length, about 5 to about 15 amino acid residues in length, about 8 to about 20 amino acid residues in length, about 8 to about 15 amino acid residues in length, about 8 to about 12 amino acid residues in length, or about 8 to about 12 amino acid residues in length). In some embodiments, the peptide is about 8 to about 12 amino acid residues in length. In some embodiments, the peptide is about 10 to about 25 amino acid residues in length. In some embodiments, the peptide is about 5 amino acid residues in length, 6 amino acid residues in length, 7 amino acid residues in length, 8 amino acid residues in length, 9 amino acid residues in length, 10 amino acid residues in length, 11 amino acid residues in length, 12 amino acid residues in length, 13 amino acid residues in length, 14 amino acid residues in length, 15 amino acid residues in length, 16 amino acid residues in length, 17 amino acid residues in length, 18 amino acid residues in length, 19 amino acid residues in length, 20 amino acid residues in length, 21 amino acid residues in length, 22 amino acid residues in length, 23 amino acid residues in length, 24 amino acid residues in length, 25 amino acid residues in length, 26 amino acid residues in length, 27 amino acid residues in length, 28 amino acid residues in length, 29 amino acid residues in length, or 30 amino acid residues in length.

The peptide can be a peptide fragment of a human protein or a peptide fragment of a non-human protein. In some examples, the human protein is expressed on a cancer cell. In some embodiments, the peptide is a peptide fragment of melanocyte differentiation antigen gp100. n some embodiments, the peptide comprises a sequence at least 80% (for example, at least about 80%, about 85%, about 90%, about 95%, about 98%, or about 99%) identical to KVPRNQDWL (GP100; SEQ ID NO: 7) or SIINFEKL (SEQ ID NO: 8; OVA) or a fragment thereof.

Examples of proteins on cancer cells include, but are not limited to, CD19, HER2, B-cell maturation antigen (BCMA), New York esophageal squamous cell carcinoma 1 (NY-ESO-1), epidermal growth factor receptor (EGFR, EGFRvIII), epithelial cell adhesion molecule (EpCAM), NKG2D ligands, Interleukin 13 receptor α2 (IL13Rα2).

In some embodiments, the human protein is associated with an autoimmune disease (see for example, Table 3). In some embodiments, the human protein is selected from the proteins listed in Table 3.

In some embodiments, the non-human protein can be a protein of a pathogen (e.g., virus, bacteria, or parasite).

In some embodiments, the composition disclosed herein comprises a peptide fragment of a viral protein of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papillomavirus, Respiratory syncytial virus, Adenovirus, Coxsackie virus, Dengue virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Lymphocytic choriomeningitis virus, Simian Immunodeficiency virus, Human T-cell Leukemia virus type-1, Hantavirus, Rubella virus, Simian Immunodeficiency virus, Human Immunodeficiency virus type-1, or Human Immunodeficiency virus type-2.

In some embodiments, the composition disclosed herein comprises a peptide fragment of a bacterial protein of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii, other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, Clostridium difficile, other Clostridium species, Yersinia enterolitica, or other Yersinia species, or Mycoplasma species.

In some embodiments, the composition disclosed herein comprises a peptide fragment of a parasite protein of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, or Entamoeba histolytica.

In some examples, the composition disclosed herein comprises a nanoparticle, an MHC molecule, and a peptide, wherein the peptide is a fragment of an influenza virus protein (e.g., NP366, PA224, PB1-703, PB1-F2, and NP55), a fragment of a human papillomavirus protein (e.g., E2 and E3), a fragment of a human immunodeficiency virus protein, a fragment of a lymphocytic choriomeningitis virus protein (e.g., GP33), a fragment of a cytomegalovirus protein, a fragment of an Epstein-Barr virus protein, or a fragment of a SARS-CoV-2 protein. In some embodiments, the peptide is NS2, NP366, PA224, PB1-703, PB1-F2, NP55, or GP33 or a fragment thereof. In some embodiments, the peptide comprises a sequence at least 80% (for example, at least about 80%, about 85%, about 90%, about 95%, about 98%, or about 99%) identical to a sequence selected from the group consisting of KAVYNFATM (SEQ ID NO: 1; GP33), ASNENMETM (SEQ ID NO: 2; NP366), SSLENFRAYV (SEQ ID NO: 3; PA224), SSYRRPVGI (SEQ ID NO: 4; PB1-703), LSLRNPILV (SEQ ID NO: 5; PB1-F2), RTFSFQLI (SEQ ID NO: 9; NS2), and RLIQNSLTI (SEQ ID NO: 6; NP55), or a fragment thereof.

It should be understood that one MHC allele can be specifically recognized by one or more peptides. Accordingly, in some examples, the composition disclosed herein comprises one or more MHC molecules and one or more peptides, wherein the MHC molecules are the same MHC allele, and wherein the one or more peptides each binds to one of the MHC molecules. In some embodiments, the one or more peptides comprise same amino acid sequence. In some embodiments, the one or more peptides comprise different amino acid sequences.

In some embodiments, the MHC class molecules disclosed herein are located on the surface of the nanoparticles disclosed herein. In some embodiments, the density of the MHC molecule on the nanoparticle is about 0.6-0.7 microgram pMHC per microgram lipids.

The term “nanoparticle” as used herein refers to a particle or structure which typically ranges from about 1 nm to about 1000 nm in size, preferably from about 50 nm to about 500 nm size, more preferably from about 50 nm to about 350 nm size, more preferably from about 100 nm to about 250 nm size.

In some embodiments, the nanoparticle has a diameter from about 1 nm to about 1000 nm. In some embodiments, the nanoparticle has a diameter less than, for example, about 1000 nm, about 950 nm, about 900 nm, about 850 nm, about 800 nm, about 750 nm, about 700 nm, about 650 nm, about 600 nm, about 550 nm, about 500 nm, about 450 nm, about 400 nm, about 350 nm, about 300 nm, about 290 nm, about 280 nm, about 270 nm, about 260 nm, about 250 nm, about 240 nm, about 230 nm, about 220 nm, about 210 nm, about 200 nm, about 190 nm, about 180 nm, about 170 nm, about 160 nm, about 150 nm, about 140 nm, about 130 nm, about 120 nm, about 110 nm, about 100 nm, about 90 nm, about 80 nm, about 70 nm, about 60 nm, about 50 nm, about 40 nm, about 30 nm, about 20 nm, or about 10 nm. In some embodiments, the nanoparticle has a diameter, for example, from about 20 nm to about 1000 nm, from about 20 nm to about 800 nm, from about 20 nm to about 700 nm, from about 30 nm to about 600 nm, from about 30 nm to about 500 nm, from about 40 nm to about 400 nm, from about 40 nm to about 300 nm, from about 40 nm to about 250 nm, from about 50 nm to about 250 nm, from about 50 nm to about 200 nm, from about 50 nm to about 150 nm, from about 60 nm to about 150 nm, from about 70 nm to about 150 nm, from about 80 nm to about 150 nm, from about 90 nm to about 150 nm, from about 100 nm to about 150 nm, from about 110 nm to about 150 nm, from about 120 nm to about 150 nm, from about 90 nm to about 140 nm, from about 90 nm to about 130 nm, from about 90 nm to about 120 nm, from 100 nm to about 140 nm, from about 100 nm to about 130 nm, from about 100 nm to about 120 nm, from about 100 nm to about 110 nm, from about 110 nm to about 120 nm, from about 110 nm to about 130 nm, from about 110 nm to about 140 nm, from about 90 nm to about 200 nm, from about 100 nm to about 195 nm, from about 110 nm to about 190 nm, from about 120 nm to about 185 nm, from about 130 nm to about 180 nm, from about 140 nm to about 175 nm, from 150 nm to 175 nm, or from about 150 nm to about 170 nm. In some embodiments, the nanoparticle has a diameter from about 100 nm to about 250 nm. In some embodiments, the nanoparticle has a diameter from about 150 nm to about 175 nm. In some embodiments, the nanoparticle has a diameter from about 135 nm to about 175 nm. The particles can have any shape but are generally spherical in shape.

A nanoparticle has a surface charge that attracts ions having opposite charge to the nanoparticle surface. Such a double layer of ions travels with the nanoparticle. Zeta potential refers to the electrostatic potential at the electrical double layer. In some embodiments, the nanoparticle disclosed herein has a zeta potential ranging from about −10 mV to about −100 mV, about −20 mV to about −100 mV, about −30 mV to about −100 mV, about −40 mV to about −100 mV, about −50 mV to about −100 mV, about −60 mV to about −100 mV, about −10 mV to about −80 mV, about −10 mV to about −70 mV, about −10 mV to about −50 mV, about −10 mV to about −30 mV, about −20 mV to about −70 mV, about −20 mV to about −40 mV, about −30 mV to about −60 mV, less than about −5 mV, less than about −6 mV, less than about −7 mV, less than about −9 mV, less than about −10 mV, less than about −11 mV, less than about −12 mV, less than about 13 mV, less than about −14 mV, less than about −15 mV, less than about −16 mV, less than about −17 mV, less than about −18 mV, less than about −19 mV, less than about −20 mV, less than about −21 mV, less than about −22 mV, less than about −23 mV, less than about −24 mV, less than about −25 mV, less than about −26 mV, less than about −27 mV, less than about −28 mV, less than −29 mV. In some embodiments, the nanoparticle disclosed herein has a zeta potential about −10 mV, about −12 mV, about −13 mV, about −14 mV, about −15 mV, about −16 mV, about −17 mV, about −18 mV, about −20 mV, about −22 mV, about −24 mV, about −26 mV, about −28 mV, about −30 mV, about −40 mV, about −41 mV, about −42 mV, about −43 mV, about −44 mV, about −45 mV, about −46 mV, about −47 mV, about −48 mV, about −49 mV, about −50 mV, about −55 mV, about −60 mV, about −70 mV, about −80 mV, about −90 mV, or about −100 mV. In some embodiments, the nanoparticle disclosed herein has a zeta potential about −15 mV to about −30 mV. In some embodiments, the nanoparticle disclosed herein has a zeta potential about −25 mV to about −35 mV.

In some embodiments, the nanoparticle is a lipid nanoparticle. The term “lipid nanoparticle” refers to a nanoparticle as described above that includes lipids and that is stable and dispersible in aqueous media. In some embodiments, the nanoparticle comprises MC3 ((6Z,9Z,28Z,31Z)-Heptatriaconta-6,9,28,31-tetraen-19-yl4-(dimethylamino)butanoate), phosphatidylcholine (1,2-distearoyl-sn-glycero-3-phosphocholin (DSPC), cholesterol, distearoyl glycerol-polyethylene glycol (DMG-PEG), and/or 1,2-Distearoyl-sn-glycero-3-phosphorylethanolamine (DSPE)-PEG. In some embodiments, the lipid nanoparticle is that described in U.S. Publication No. 20200315967, which is incorporated by reference in its entirety.

The MHC molecule disclosed herein can be linked to the nanoparticle through a linker. In some examples, the linker comprises maleimide. The maleimide-containing linker enables anchoring of the proteins (e.g., an MHC molecule). In some embodiments, the linker comprises 1,2-Distearoyl-sn-glycero-3-phosphorylethanolamine (DSPE), polyethylene glycol (PEG), and/or maleimide.

Accordingly, in some aspects, disclosed herein is a composition comprising
a nanoparticle;
a MHC molecule; and
a peptide, wherein the peptide binds to the MHC molecule, wherein the MHC molecule and the peptide are located on the surface of the nanoparticle, and wherein the MHC molecule is linked to the nanoparticle through a linker, wherein the linker comprises 1,2-Distearoyl-sn-glycero-3-phosphorylethanolamine (DSPE), polyethylene glycol (PEG), and/or maleimide.

In some embodiments, the nanoparticle disclosed herein further comprises an agent. In some embodiments, the agent is encapsulated inside the nanoparticle. In some embodiments, the agent is a nucleic acid. In some embodiments, the nucleic acid is RNA or DNA. In some embodiments, the nucleic acid encodes a chimeric antigen receptor (CAR), a CRISPR/Cas9 endonuclease, a costimulatory molecule, and/or a cytokine (e.g., IL-2, IL-10, or IL-15). In some embodiments, the nucleic acid comprises a sequence of one of SEQ ID NOS: 26-27 and 31-33.

Also disclosed herein is a pharmaceutical composition comprising the nanoparticle disclosed herein and a pharmaceutically acceptable carrier.

Also disclosed herein is a method of treating a cancer is a subject in need, comprising administering to the subject a therapeutically effective amount of the composition disclosed herein. In some embodiments, the composition targets anti-cancer T cells. In some embodiments, the anti-cancer T cell is a CD8 T cell. In some embodiments, the composition comprises a nanoparticle comprising a nucleic acid encoding a CAR.

Also disclosed herein is a method of treating an autoimmune disorder in a subject in need, comprising administering to the subject a therapeutically effective amount of the composition disclosed herein. In some embodiments, the composition targets a regulatory T cell. In some embodiments, the composition comprises a nanoparticle comprising a nucleic acid encoding IL-10 and/or TGF-β.

Dosing frequency for the therapeutic agent disclosed herein, includes, but is not limited to, at least once every 12 months, once every 11 months, once every 10 months, once every 9 months, once every 8 months, once every 7 months, once every 6 months, once every 5 months, once every 4 months, once every 3 months, once every two months, once every month; or at least once every three weeks, once every two weeks, once a week, twice a week, three times a week, four times a week, five times a week, six times a week, or daily. In some embodiments, the interval between each administration is less than about 4 months, less than about 3 months, less than about 2 months, less than about a month, less than about 3 weeks, less than about 2 weeks, or less than less than about a week, such as less than about any of 6, 5, 4, 3, 2, or 1 day. In some embodiments, the dosing frequency for the nanoparticle composition includes, but is not limited to, at least once a day, twice a day, or three times a day. In some embodiments, the interval between each administration is less than about 48 hours, 36 hours, 24 hours, 22 hours, 20 hours, 18 hours, 16 hours, 14 hours, 12 hours, 10 hours, 9 hours, 8 hours, or 7 hours. In some embodiments, the interval between each administration is less than about 24 hours, 22 hours, 20 hours, 18 hours, 16 hours, 14 hours, 12 hours, 10 hours, 9 hours, 8 hours, 7 hours, or 6 hours. In some embodiments, the interval between each administration is constant. For example, the administration can be carried out daily, every two days, every three days, every four days, every five days, or weekly. Administration can also be continuous and adjusted to maintaining a level of the compound within any desired and specified range.

Dosages are typically modified according to the characteristics of the subject (weight, gender, age, etc.), severity of disease, specifics and purity of the active agent to be administered, route of administration, nature of the formulation, and numerous other factors. Generally, the active agent (e.g., the encapsulated mRNA) is administered to the subject at a dosage ranging from 0.1 μg/kg body weight to 500 mg/kg body weight. In some embodiments, the active agent is administered to the subject at a dosage of from 10 μg/kg to 500 mg/kg, from 10 μg/kg to 100 mg/kg, from 10 μg/kg to 10 mg/kg, from 10 μg/kg to 1 mg/kg, from 10 μg/kg to 500 μg/kg, or from 10 μg/kg to 100 μg/kg body weight. The dosage of administration for the active agent disclosed herein can be from about 0.01 mg/kg body weight to about 100 mg/kg body weight, about 0.01 mg/kg body weight to about 10 mg/kg body weight, about 0.01 mg/kg body weight to about 1 mg/kg body weight, or about 0.01 mg/kg body weight to about 0.1 mg/kg body weight. Dosages above or below the range cited above may be administered to the individual patient if desired.

Also disclosed herein is a method of modifying an antigen-specific T cell, wherein said method comprises contacting the antigen-specific T cell with the composition disclosed herein.

Also disclosed herein is a method of delivering a nucleic acid to an antigen specific T cell, wherein said method comprises contacting the antigen-specific T cell with the composition disclosed herein.

In some embodiments, the T cell is an activated T cell.

In some embodiments, the composition is internalized by the antigen-specific T cell. In some embodiments, the agent is encapsulated inside the nanoparticle. In some embodiments, the agent is a nucleic acid. In some embodiments, the nucleic acid is RNA or DNA. In some embodiments, the nucleic acid is mRNA. In some embodiments, the nucleic acid encodes a chimeric antigen receptor (CAR), a CRISPR/Cas9 endonuclease, a costimulatory molecule, and/or a cytokine (e.g., IL-2, IL-10, or IL-15). In some embodiments, the nucleic acid comprises a sequence of one of SEQ ID NOS: 26-27 and 31-33. The APNs disclosed herein can be used for any CARs (e.g., BCMA CAR) other than mouse and human CD19 CAR shown herein.

In some embodiments, the antigen-specific T cell makes contact with the composition in vivo or ex vivo.

In some embodiments, the sacrificial peptide comprises a photolabile group, such that upon photocleavage by UV light, the sacrificial peptide dissociates to allow an exchange peptide to bind to the MHCI presentation groove.

In some aspects, disclosed herein is a composition comprising a nanoparticle and a major histocompatibility complex (MHC) molecule, wherein the MHC molecule comprises a heavy chain that comprises a C-terminal cysteine.

In some embodiments, the MHC molecule comprises a MHC class 1 molecule or a MHC class 2 molecule. In some embodiments, the heavy chain comprises a sequence at least 60% identical to SEQ ID NO: 18, 19, or 20. In some embodiments, the MHC molecule is attached to an epitope.

### High Throughput Methods

In some embodiments, disclosed herein is a high throughput method of creating a composition that comprises a nanoparticle, a major histocompatibility complex (MHC) molecule, and a peptide, said method comprising:
attaching the MHC molecule to a sacrificial peptide;
contacting the MHC molecule with a lipid micelle to create a first MHC micelle;
contacting the first MHC micelle with the peptide;
applying a UV light to the first MHC micelle and the peptide to create a second MHC micelle; and
creating the composition by contacting the second MHC micelle with the nanoparticle.

In some embodiments, the MHC molecule comprises an MHC class I molecule or an MHC class II molecule. In some examples, the MHC molecule disclosed herein is a human MHC molecule. The human MHC molecule is also called the “HLA” (human leukocyte antigen). In some embodiments, the composition disclosed herein comprises an MHC class I molecule or an MHC class II molecule. In some embodiments, the human MHC molecule is HLA-A*01:01, HLA-A*02:01, HLA-A*03:01, HLA-A*07:02, HLA-A*11:01, or HLA-A*24:02. In some embodiments, the MHC molecule disclosed herein is a mouse MHC molecule. In some embodiments, the mouse MHC molecule is H2-D^{b}, H2-K^{b}, or H2-L^{b}. In some examples, the heavy chain of the MHC class I molecule can comprises a C-terminal cysteine. In some embodiments, the heavy chain comprises a sequence at least 60% identical to one of SEQ ID NOS: 23-25 or a fragment thereof.

In some embodiments, the sacrificial peptide comprises a UV sensitive peptide. In some embodiments, the sacrificial peptide comprises a sequence selected from SEQ ID NOs: 10-17 or a fragment thereof.

In some embodiments, the first MHC micelle is contacted with a peptide fragment of a human protein or a peptide fragment of a non-human protein. In some examples, the human protein is expressed on a cancer cell. In some embodiments, the peptide is a peptide fragment of melanocyte differentiation antigen gp100. n some embodiments, the peptide comprises a sequence at least 80% (for example, at least about 80%, about 85%, about 90%, about 95%, about 98%, or about 99%) identical to KVPRNQDWL (GP100; SEQ ID NO: 7) or SIINFEKL (SEQ ID NO: 8; OVA) or a fragment thereof. In some examples, the human protein is associated with an autoimmune disease.

In some embodiments, the peptide is a peptide fragment of a non-human protein, which is, for example, a protein of a pathogen (e.g., virus, bacteria, or parasite). In some examples, the non-human protein is an influenza virus protein (e.g., NP366, PA224, PB1-703, PB1-F2, and NP55), a human papillomavirus protein (e.g., E2 and E3), a human immunodeficiency virus protein, a lymphocytic choriomeningitis virus protein (e.g., GP33), a cytomegalovirus protein, an Epstein-Barr virus protein, or a SARS-CoV-2 protein. In some embodiments, the peptide is NS2, NP366, PA224, PB1-703, PB1-F2, NP55, or GP33 or a fragment thereof. In some embodiments, the peptide comprises a sequence at least 80% (for example, at least about 80%, about 85%, about 90%, about 95%, about 98%, or about 99%) identical to a sequence selected from the group consisting of KAVYNFATM (SEQ ID NO: 1; GP33), ASNENMETM (SEQ ID NO: 2; NP366), SSLENFRAYV (SEQ ID NO: 3; PA224), SSYRRPVGI (SEQ ID NO: 4; PB1-703), LSLRNPILV (SEQ ID NO: 5; PB1-F2), RTFSFQLI (SEQ ID NO: 9; NS2), and RLIQNSLTI (SEQ ID NO: 6; NP55), or a fragment thereof.

It should be understood that one MHC allele can be specifically recognized by one or more peptides. Accordingly, in some examples, the method comprises contacting the first MHC micelle with one or more peptides. In some embodiments, the one or more peptides comprise same amino acid sequence. In some embodiments, the one or more peptides comprise different amino acid sequences.

In some embodiments, the nanoparticle is a lipid nanoparticle. The term “lipid nanoparticle” refers to a nanoparticle as described above that includes lipids and that is stable and dispersible in aqueous media. In some embodiments, the nanoparticle comprises MC3, phosphatidylcholine (1,2-distearoyl-sn-glycero-3-phosphocholin (DSPC), cholesterol, distearoyl glycerol-polyethylene glycol (DMG-PEG), and/or 1,2-Distearoyl-sn-glycero-3-phosphorylethanolamine (DSPE)-PEG. In some embodiments, the lipid nanoparticle is that described in U.S. Publication No. 20200315967, which is incorporated by reference in its entirety.

The MHC molecule disclosed herein can be linked to the nanoparticle through a lipid. In some examples, the lipid comprises maleimide. The maleimide-containing lipid enables anchoring of the proteins (e.g., an MHC molecule). In some embodiments, the lipid linking the MHC molecule to the nanoparticle comprises 1,2-Distearoyl-sn-glycero-3-phosphorylethanolamine (DSPE), polyethylene glycol (PEG), and/or maleimide.

In some embodiments, the MHC molecule on the second MHC micelle contacts the nanoparticle in a ratio of about 1:10, 1:20, 1:30, 1:40, 1:50, 1:60, 1:80, or 1:100 (molar ratio of maleimide:D-Lin-MC3-DMA). In some embodiments, the ratios include about: 50% ionizable lipid Dlin-MC3-DMA, 10% DSPC, 38% Cholesterol, 0.5% DSPE-PEG and 1.5% DMG-PEG.

In some embodiments, the method further comprises a step of encapsulating an agent into the nanoparticle. In some embodiments, the agent is encapsulated in the interior of the nanoparticle. In some embodiments, the agent is a nucleic acid. In some embodiments, the nucleic acid is RNA or DNA. In some embodiments, the nucleic acid is an mRNA. In some embodiments, the nucleic acid encodes a chimeric antigen receptor (CAR), a CRISPR/Cas9 endonuclease, a costimulatory molecule, and/or a cytokine (e.g., IL-2, IL-10, or IL-15). In some embodiments, the nucleic acid comprises a sequence of one of SEQ ID NOS: 26-27 and 31-33. The APNs disclosed herein can be used for any CARs (e.g., BCMA CAR) other than mouse and human CD19 CAR shown herein.

In some aspects, disclosed herein is a high throughput method of creating a composition that comprises a nanoparticle, a major histocompatibility complex (MHC) molecule, and an epitope, comprising:
a. attaching the MHC molecule to a sacrificial peptide;
b. contacting the MHC molecule of step a with a lipid micelle to create a first MHC micelle;
c. contacting the first MHC micelle with the epitope;
d. applying a UV light to the first MHC micelle and the epitope to create a second MHC micelle; and
e. creating the composition by incubating the second MHC micelle with the nanoparticle.

In some embodiments, the MHC molecule comprises a heavy chain that comprises a C-terminal cysteine. In some embodiments, the heavy chain comprises a sequence at least 60% identical to SEQ ID NO: 18, 19, or 20. In some embodiments, the sacrificial peptide comprises a UV-labile amino acid.

In some aspects, disclosed herein is a method for modifying an antigen-specific T cell, comprising contacting the antigen-specific T cell with a composition that comprises a nanoparticle, a major histocompatibility complex (MHC) molecule, and an agent.

In some embodiments, the agent modifies the antigen-specific T cell. In some embodiments, the agent is encapsulated inside the nanoparticle.

### EXAMPLES

The following examples are set forth below to illustrate the compositions, methods, and results according to the disclosed subject matter. The examples are not intended to be inclusive of all aspects of the subject matter disclosed herein, but rather to illustrate representative methods and results. These examples are not intended to exclude equivalents and variations of the present invention which are apparent to one skilled in the art.

### Example 1. In Vivo mRNA Delivery to Virus-Specific T Cells by Light-Induced Ligand Exchange of MHC Class I Antigen-Presenting Nanoparticles

Simultaneous delivery of mRNA to multiple populations of antigen (Ag)-specific CD8⁺ T cells is challenging given the diversity of peptide epitopes and polymorphism of class I major histocompatibility complexes (MHCI). Ag-presenting nanoparticles (APNs) for mRNA delivery using pMHCI molecules were refolded with photocleavable peptides to allow rapid ligand exchange by UV light and site-specifically conjugated with a lipid tail for postinsertion into preformed mRNA lipid nanoparticles. Across different TCR transgenic mouse models (P14, OT-1, and Pmel), UV-exchanged APNs bound and transfected their cognate Ag-specific CD8 T cells equivalent to APNs produced using conventionally refolded pMHCI molecules. In mice infected with PR8 influenza, multiplexed delivery of UV-exchanged APNs against three immunodominant epitopes led to ˜50% transfection of a VHH mRNA reporter in cognate Ag-specific CD8 T cells. These data show that UV-mediated peptide exchange can be used to rapidly produce APNs for mRNA delivery to multiple populations of Ag-specific T cells in vivo.

### Introduction

Antigen (Ag)-specific CD8⁺ T cells express T cell receptors (TCRs) that recognize processed peptide Ags bound to major histocompatibility complex class I (MHCI) molecules expressed on the cell surface. The TCR-peptide MHCI (pMHCI) interaction forms the basis for the exquisite specificity of CD8⁺ T cell recognition and their cytotoxic activity against target cells bearing cognate pMHCIAgs. This central mechanism has driven increasing interest indelivery approaches that can target and modulate T cells for immunotherapy. Recent studies include delivery of immunomodulatory molecules (e.g., transforming growth factor-3 inhibitors) using nanoparticles decorated with antibodies against T cell surface markers, including CD3 and PD-1, to enhance effector functions within the tumor microenvironment. Programming endogenous CD3⁺ or CD8⁺ T cells with polymer/lipid nanoparticles (LNPs) loaded with nucleic acids [e.g., CD45 small interfering RNA (siRNA) and chimeric Ag receptor (CAR)-encoded DNA] has shown potential to silence target genes in T cells or for in situ manufacturing of CAR T cells. The ability to target Ag-specific T cells offers opportunities to selectively augment disease-relevant T cell subsets (e.g., viral or tumor Ag-specific T cells) in vivo while maintaining homeostasis and self-tolerance of the immune system. To target Ag-specific T cells in vivo, strategies include engineered human pMHCI [human leukocyte Ag (HLA)]-Fc fusion dimers to expand human papillomavirus (HPV)-specific CD8⁺ T cells against HPV-associated malignancies or track virus-specific CD8⁺T cells by immune positron emission tomography imaging, artificial Ag-presenting cells composed of pMHCI on nanoparticles or engineered red blood cells to activate Ag-specific T cells and enhance their effector function for cancer treatment, tumor-targeting antibodies to deliver viral peptides that are cleaved by tumor proteases and then loaded onto MHCI on the tumor cell surface to redirect virus-specific T cells against tumors, and nanoparticles deco-rated with pMHCII molecules to reprogram autoantigen-reactive CD4⁺ T cells into disease-suppressing regulatory T cells (T_{reg}). These studies highlight the broad applications of in vivo delivery to Ag-specific T cells.

Despite considerable interest, however, multiplexed delivery to distinct populations of Ag-specific T cells remains challenging owing to the complexity of the immune response. For example, more than 500 severe acute respiratory syndrome coronavirus 2 CD8⁺ T cell epitopes that are restricted across 26 HLA class I alleles have been described so far. Conventionally, pMHCI molecules are expressed by individual refolding reactions to assemble three components—an invariant light chain, a polymorphic heavy chain, and a peptide ligand—into the heterotrimeric structure of endogenous pMHCI molecules. This serial process precludes production of large pMHCI libraries until the development of peptide exchange strategies mediated by ultraviolet (UV) light, temperature, dipeptides, or chaperone proteins. With UV light mediated peptide exchange, the heavy and light chains are refolded with a sacrificial peptide containing a photolabile group, such that upon photocleavage by UV light, the sacrificial peptide dissociates to allow an exchange peptide to bind to the MHCI presentation groove. For a particular MHC allele, a single batch of UV-sensitive pMHCI molecules can be conventionally refolded and then used to produce hundreds of pMHCI molecules carrying different peptides in one step. For example, pMHCI tetramer libraries with >1000 peptide specificities have been described for the detection of neoAg-specific T cells.

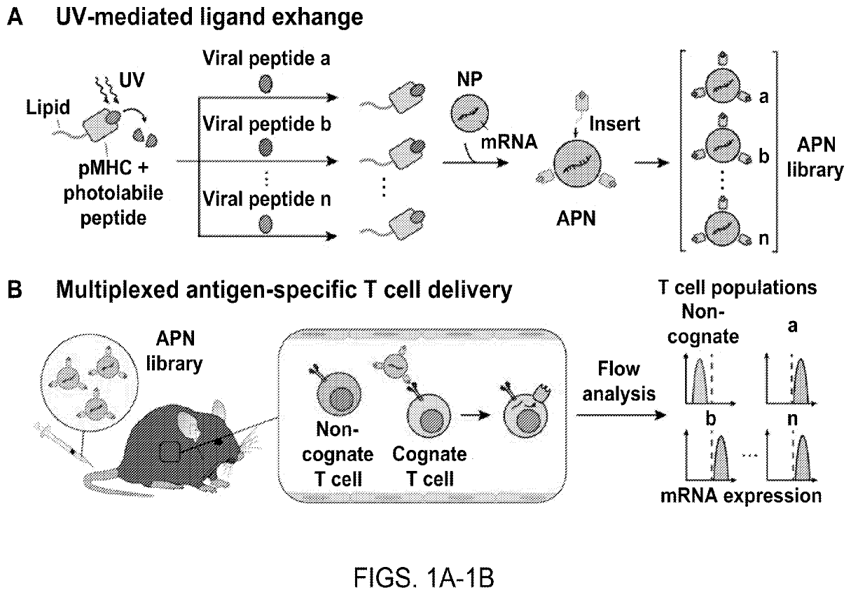

Here, Ag-presenting LNPs (APNs) were synthesized using UV light-mediated ligand exchange for mRNA delivery to multiple influenza-specific CD8⁺ T cells (FIG. 1). This approach increases the precision of T cell delivery compared to antibodies (e.g., CD3 and CD8), is rapidly scalable to different peptide epi-topes, and, through mRNA delivery, can enable a range of applications from in situ manufacturing of T cell therapies to genome editing and regulation. This method used UV light-mediated ligand exchange to produce a panel of pMHCI molecules from a sacrificial pMHCI precursor that was site-specifically modified with a lipid tail. This allowed postinsertion after peptide exchange to preformed LNPs, which was formulated on the basis of a similar MC3-based composition as the first U.S. Food and Drug Administration-approved siRNA drug (ONPATTRO), encapsulating a model mRNA reporter encoding a camelid VHH antibody. Decorating APNs with conventionally refolded or peptide-exchanged pMHCI molecules targeted and transfected Ag-specific CD8⁺ T cells in multiple TCR transgenic mouse models in vivo (P14, Pmel-1, and OT-1) regardless of the MHC allotype (H2-D^{b} for P14 and Pmel and H2-K^{b} for OT-1). In a mouse model of recombinant influenza A viral infection (A/Puerto Rico/8/34 H1N1 modified with GP33 Ag, abbreviated as PR8-GP33), intravenous administration of a three-plex cocktail of peptide-exchanged APNs (NP366/D^{b}, PA224/D^{b}, and GP33/D^{b}) resulted in the simultaneous transfection of the top three immunodominant PR8-GP33-specific T cell populations that was significantly more efficient compared to other major cell populations in the spleen and liver. This data shows that UV light-mediated peptide exchange allows for parallel production of APNs for functional mRNA delivery to multiple Ag-specific CD8⁺ T cell populations in vivo.

### Results

Antigen-Presenting Nanoparticles (APNs) Bind to Ag-Specific T Cells and Induce Internalization for mRNA Transfection.

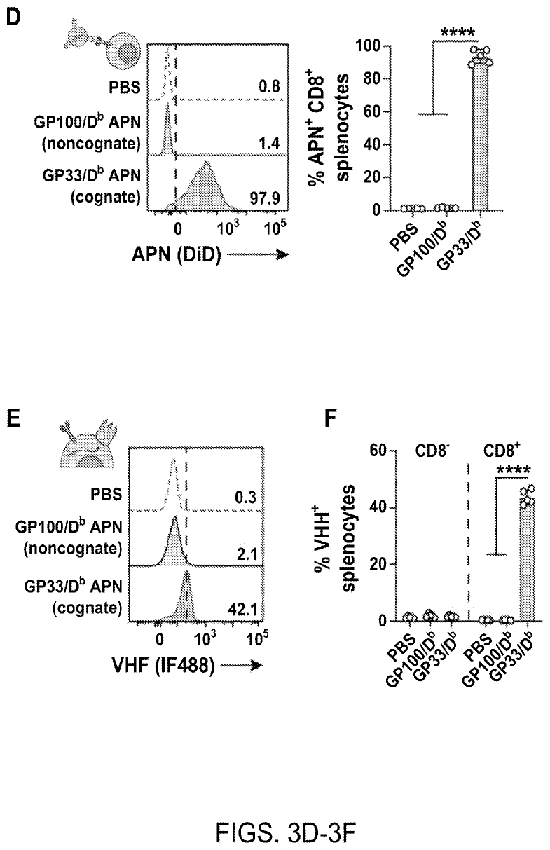

The insertion of derivatives modified with lipids to preformed nanoparticles is a well-established approach to decorate nanoparticles with ligands that are stabilized by hydrophobic interactions. For example, post insertion is commonly used to PEGylate liposomes or LNPs using polyethylene glycol (PEG) polymers derivatized with lipid tails. Expression of recombinant pMHCI molecules with a site-specific handle for conjugation of a lipid was developed such that the complex serves as the starting point for peptide exchange before post insertion to LNPs (FIG. 1A). Lymphocytic choriomeningitis virus (LCMV) Ag GP33/D^{b} (KAVYNFATM/D^{b}; SEQ ID NO: 1) with a C-terminal cysteine in the heavy chain to prevent disruption of native disulfide bonds and retain proper pMHCI orientation for TCR recognition was expressed and refolded. These were then reacted with DSPE-PEG2000-maleimide to generate lipid-modified GP33/D^{b} molecules. In parallel, synthesized MC3-based LNPs encapsulating enhanced green fluorescent protein (eGFP) mRNA by microfluidic mixers were characterized by an average diameter of 93.85 nm and a zeta potential of −30.20±0.5 mV by dynamic light scattering (FIGS. 6A and 6B). Postinsertion of lipid-modified GP33/D^{b} pMHCI molecules did not appreciably increase LNP size nor alter the zeta potential (107.9±7.34 nm and −22.73±4.7 mV, respectively) (FIGS. 6B and 6C) Successful postinsertion across various APN formulations was confirmed using bicinchoninic acid (BCA) protein quantification (Table 4). eGFP mRNA concentration was also assessed using the RiboGreen assay and found that bare LNPs and APNs were comparable (80.20±0.50% to 73.62±0.58%, respectively) (FIG. 6C).

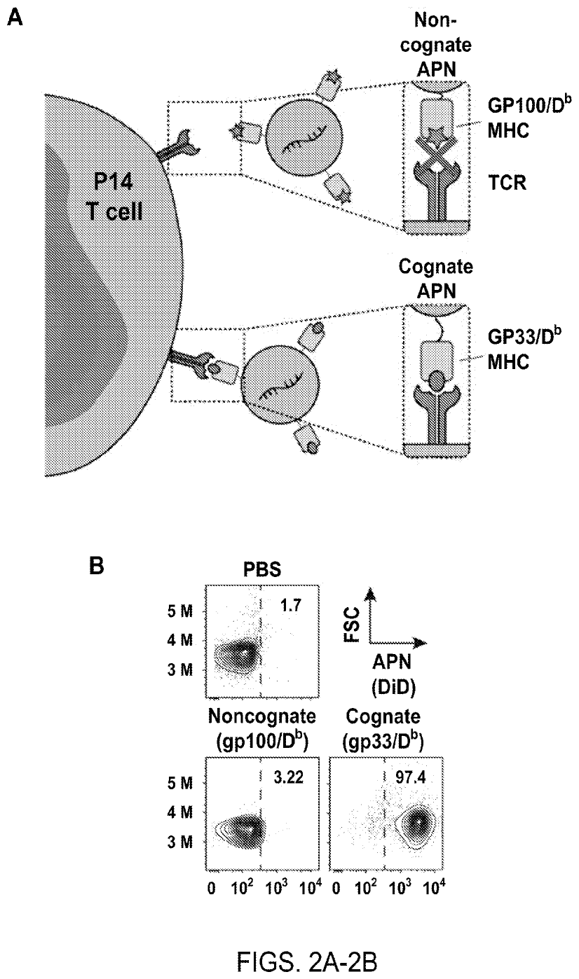

Whether GP33/D^{b} APNs can selectively bind to their cognate CD8⁺ T cells isolated from TCR transgenic P14 mice whose CD8⁺ T cells express a TCR that specifically recognizes the LCMV GP33/D^{b} Ag (FIG. 2A) was tested. It was found that GP33/D^{b} APNs bound to ˜97% of P14 CD8⁺ T cells, whereas noncognate GP100/D^{b}(KVPRNQDWL/D^{b}; SEQ ID NO: 7) APNs showed minimal staining (3.22%) (FIG. 2B). Further testing of APN binding using H2-K^{b} restricted OVA/Kb (SIINFEKL/K^{b}; SEQ ID NO: 8) APNs showed similar Ag-specific binding when co-incubated with their cognate CD8⁺ T cells isolated from OT-1 transgenic mice compared to noncognate NS2/K^{b}(RTFSFQLI/K; SEQ ID NO: 9) APNs (FIG. 2C). The observed 10% cross-reactivity in NS2/K^{b} APNs to OT-1 CD8⁺ splenocytes is due to the binding affinity of H2-K^{b} MHC to CD8 co-receptors on CD8⁺ T cells. The binding of APNs to T cells to induce internalization by T cells were investigated given that pMHCI multimers are known to be rapidly taken up by T cells through TCR clustering and receptor-mediated endocytosis at physiological temperatures. The fate of APNs after engaging cognate T cells at 37° C. compared to 4° C. were studied, which is typically used for pMHCI multimer staining to minimize T cell activation and TCR internalization. DiD-labeled OVA/Kb APNs were incubated with OT-1 CD8⁺ T cells at 4° and 37° C., followed by an acidic wash to strip uninternalized APNs bound to the TCRs on T cell surface. DiD fluorescence was found to be decreased for cells incubated at 4° C., indicating that APNs remained on the cell surface before acid wash (FIGS. 2D and 2E). For cells treated at 37° C., however, no change in fluorescence was observed, showing efficient T cell internalization of APNs. By contrast, no binding and internalization of noncognate GP100/D^{b} APNs by OT-1 CD8⁺ T cells was found under all conditions (FIG. 2E). P14 splenocytes were incubated with GP33/D^{b} APNs loaded with eGFP mRNA to determine whether pMHCI-induced TCR internalization results in functional mRNA delivery to T cells. A dose-dependent eGFP expression was observed in APN-transfected CD8⁺ T cells [mean fluorescent intensity (MFI), 397 and 506 for 1- and 2-μg mRNA doses, respectively] in contrast to splenocytes treated with phosphate-buffered saline (PBS; MFI, 153) or free mRNA (MFI, 118) (FIG. 2F). Collectively, these results demonstrate that cognate APNs target, bind, and induce T cell uptake for functional mRNA delivery in vitro.

### APNs Transfect Ag-Specific T Cells in TCR Transgenic Mice

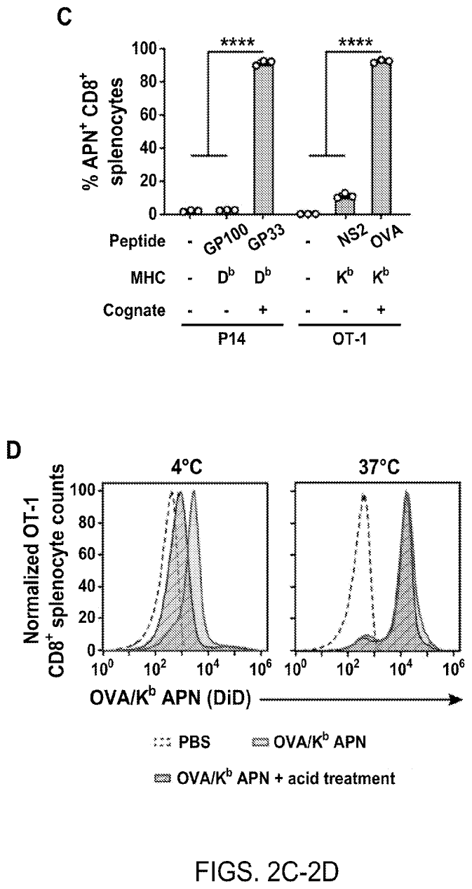

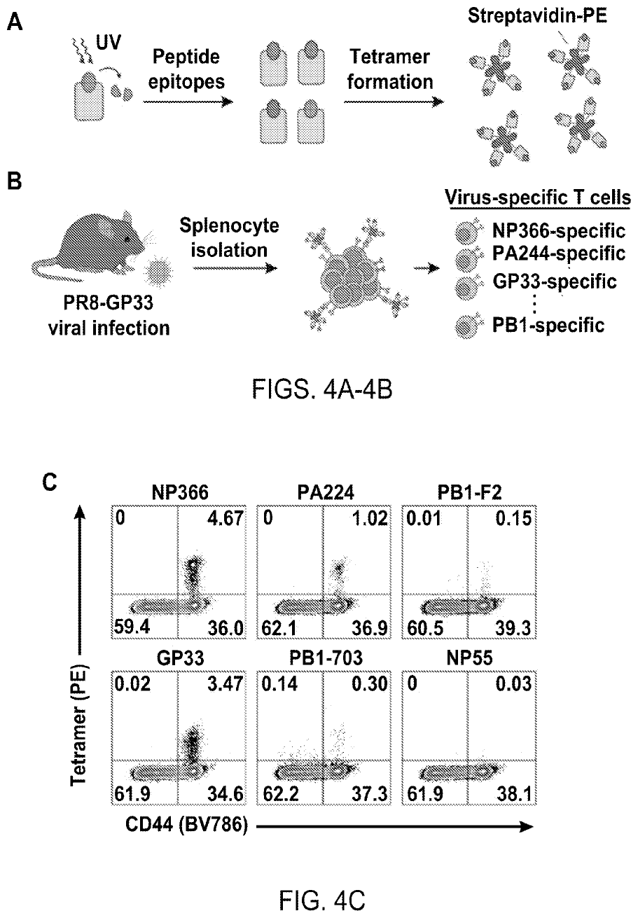

Next, quantification of in vivo biodistribution and transfection efficiency was performed for GP33/D^{b} APNs in TCR transgenic P14 mice (FIG. 3A). Functional delivery to major organs was tested using APNs loaded with firefly luciferase (Fluc) mRNA (FIGS. 3B and 3C) and found significantly higher luminescence in the spleens isolated from mice treated with GP33/D^{b} APNs compared to noncognate GP100/D^{b} APNs. Notably, no significant difference was observed in the other major organs between the two groups. P14 splenocytes were harvested 24 hours after infusion of DiD-labeled APNs encapsulating VHH mRNA and observed that cognate GP33/D^{b} APNs targeted >95% of P14 CD8⁺ T cells, while GP100/D^{b} APN controls resulted in <2% binding, as quantified by DiD fluorescence (FIG. 3D). To quantify functional delivery, mRNA encoding glycosylphosphatidylinositol (GPI) membrane-anchored VHH was used as a reporter gene, as it has been shown to achieve durable surface expression (>28 days) that can be detected by immunofluorescence staining with anti-VHH antibodies. Therefore, surface expression of VHH was stained and found that GP33/D^{b} APNs resulted in significantly higher transfection efficiency compared to its non-cognate counterpart (˜40% versus <2%, respectively; FIGS. 3E and 3F). Notably, negligible transfection (<2%) of CD8⁻ splenocytes was observed in mice treated with either GP33/D^{b} APNs or GP100/D^{b} APNs. Combined with the organ level result by IVIS imaging (FIGS. 3B and 3C), the data demonstrated that the observed Fluc luminescence in the spleen was mainly from the transfection of cognate CD8⁺ splenocytes. The results were further confirmed in a different model using Pmel mice that have been engineered to express the cognate TCR against GP100/D^{b}. Similar to the results in P14 mice, ˜30 to 40% in vivo transfection of Pmel CD8⁺ splenocytes treated with GP100/D^{b} APNs was observed compared to ˜3% transfection with GP33/D^{b} noncognate APNs (FIG. 7). Together, these results demonstrate that APNs enableT cell targeting and functional mRNA delivery in an Ag-specific manner.

### APNs Synthesized by UV-Mediated Ligand Exchange Transfect T Cells Equivalently to Folded APNs

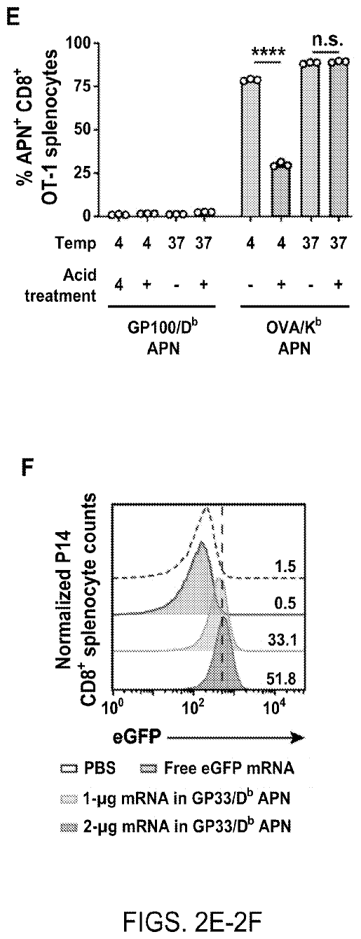

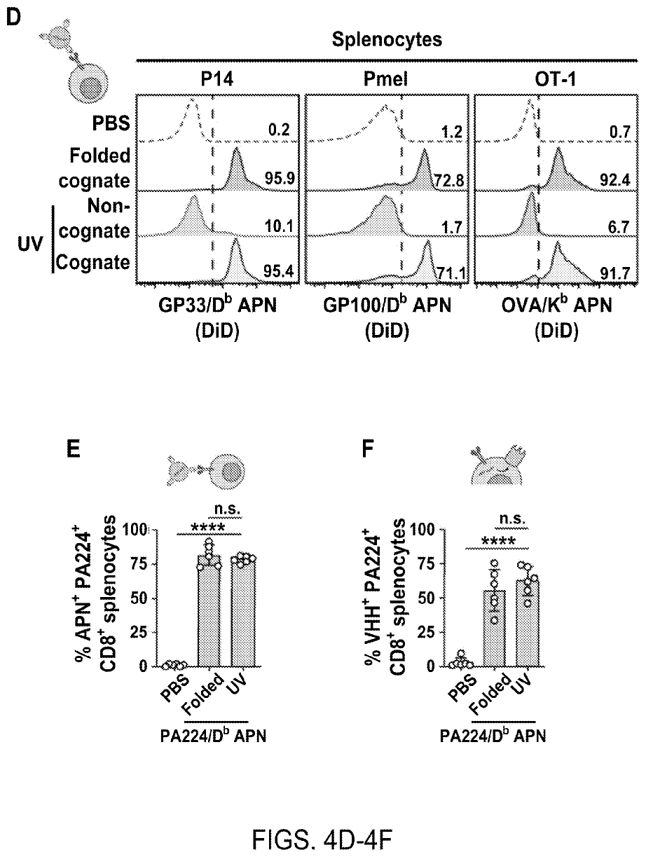

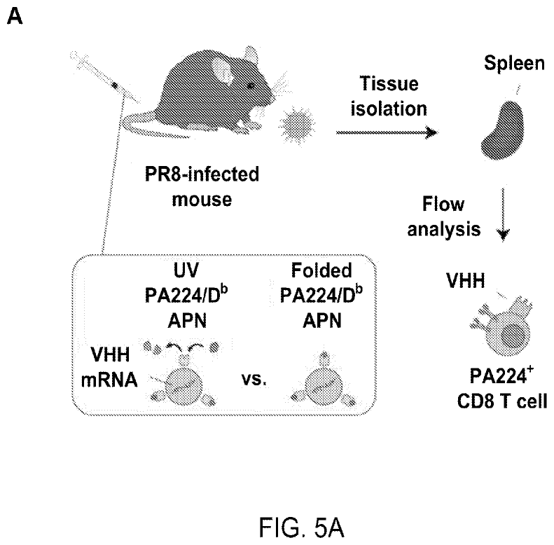

Sacrificial peptides that contain a photolabile amino acid and stabilize the MHCI complex during refolding have been previously developed for prevalent alleles including H2-D^{b} and H2-K^{b} in mice. UV-mediated peptide exchange was validated by comparing staining of P14 splenocytes using fluorescent GP33/D^{b} tetramers where the pMHCI monomers were either produced by peptide exchange from ASNENJETM/D^{b} (SEQ ID NO: 10) (J represents photocleavable amino acid) or conventionally refolded. It was first found that tetramers formed with UV-labile peptide present on H2-D^{b} MHCI did not cause any nonspecific binding to CD8⁺ splenocytes isolated from P14 and Pmel mice (FIG. 8). After UV-mediated peptide exchange into GP33 peptides, UV-exchanged tetramers showed comparable staining of CD8⁺ P14 splenocytes to folded GP33/D^{b} tetramers (>95%), whereas noncognate GP100/D^{b} tetramers produced by refolding or UV exchange resulted in minimal binding (FIG. 9). UV-exchanged tetramers to detect endogenous immune responses were further tested where T cells have a broad range of Ag specificity and binding affinities to their cognate Ags. To do this, the well-characterized mouse model of influenza virus PR8 (A/PR/8/34) modified to express the LCMV GP33 Ag (PR8-GP33) was used. Infection of mice with PR8-GP33 leads to CD8⁺ T cell responses against at least 16 PR8-derived peptide epitopes including NP366 (ASNENMETM/D^{b}; SEQ ID NO: 2), PA224 (SSLENFRAYV/D^{b}; SEQ ID NO: 3), PB1-703 (SSYRRPVGI/K^{b}; SEQ ID NO: 4), PB1-F2 (LSLRNPILV/D^{b}; SEQ ID NO: 5), and NP55 (RLIQNSLTI/D^{b}; SEQ ID NO: 6), as well as against GP33, which served as a positive control epitope. A panel of pMHCI tetramers against these six epitopes by peptide exchange (FIGS. 4A and AB) were produce and Ag-specific splenic T cell responses 10 days after infection were validated (FIG. 4C).

To integrate UV exchange for APN production, a panel of three UV-exchanged APNs inserted with GP33/D^{b}, GP100/D^{b}, and OVA/Kb pMHCI molecules was synthesized to compare with APNs inserted with pMHCI molecules synthesized using the conventional refolding protocol. In splenocytes isolated from three strains of transgenic mice (P14, Pmel, and OT-1), it was found that cognate UV-exchanged APNs bound to CD8⁺ T cells similar to the folded APNs (FIG. 4D). By contrast, only minimal background staining from the noncognate control APNs was observed. Last, tests to determine whether the UV-exchanged APNs can target and transfect virus-specific T cells in vivo using PR8-infected mice was conducted. To do this, PR8-infected mice were injected intravenously with DiD-labeled PA224/D^{b} APNs. At 24 hours after injection, consistent with the in vitro staining results (FIG. 4D), both folded and UV-exchanged PA224/D^{b} APNs targeted ˜80% of PA224-specific T cells (FIG. 4E) and resulted in comparable transfection efficiency of the model VHH mRNA (FIG. 4F).

### Multiplexed T Cell Transfection Using a Mouse Model of PR8 Flu Infection

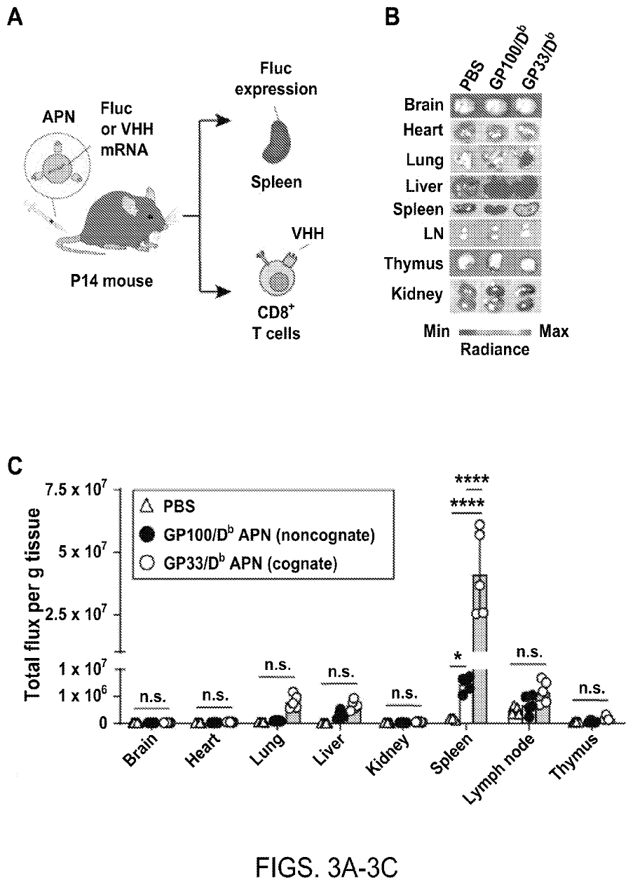

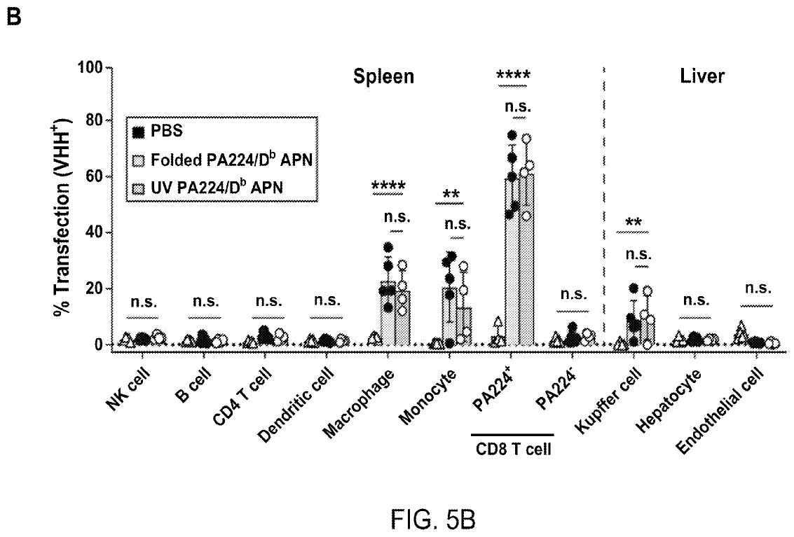

Antibodies against T cell surface markers, including CD3 and CD8, have been used to target polymeric nanoparticles to T cells in vivo irrespective of Ag specificity. Therefore, the ability of APNs to transfect virus-specific T cells was examined compared to noncognate cell populations (FIG. 5A). Analyses were focuses on liver and spleen, as these were the major organs that showed APN accumulation after intravenous administration (FIG. 10). Flow cytometry analysis of the major cell types [natural killer (NK) cells, B cells, CD4 T cells, dendritic cells, macrophages, monocytes, PA224⁺ flu-specific CD8 T cells, noncognate PA224⁻ CD8 T cells, Kupffer cells, hepatocytes, and endothelial cells] revealed that APNs preferentially transfected flu virus-specific T cells (PA224⁺ CD8 T cells, 59.46±11.81%) compared to noncognate T cells (PA224⁻ CD8 T cells, 2.63±2.17%), which comprise a population diversity of approximately 10⁶ to 10⁸ (FIG. 5B). Transfection was also observed by the reticuloendothelial system, including monocytes and macrophages in the spleen and Kupffer cells in the liver, but at significantly lower transfection efficiency than that of PA224-specific CD8 T cells (****P<0.0001). Compared to cohorts that received PBS, mice that were given folded or UV-exchanged PA224/D^{b} APNs resulted in similar transfection levels across all cell populations studied, supporting their equivalency.

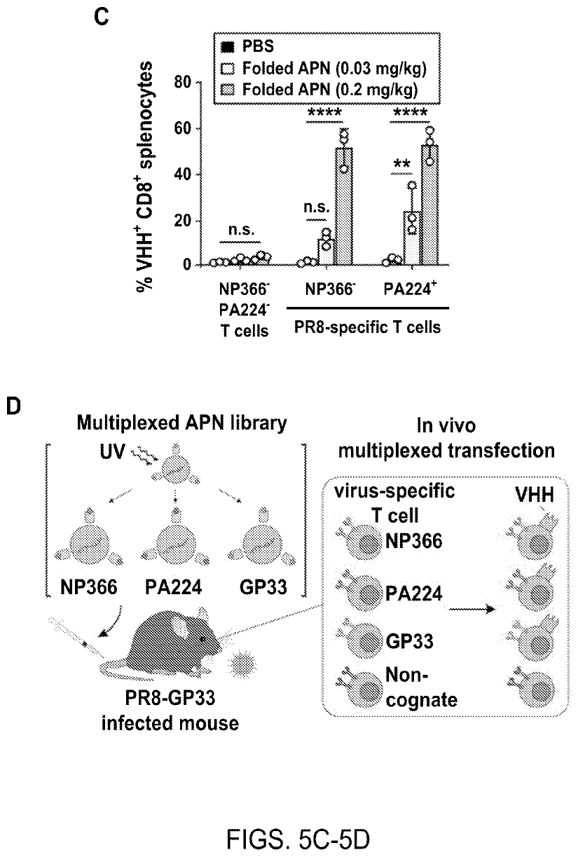

To demonstrate simultaneous transfection of distinct Ag-specific T cell populations in vivo, a mixture of DiD-labeled, conventionally refolded NP366/D^{b} and PA224/D^{b} APNs was administered to PR8-infected mice at an mRNA dose of 0.1 and 0.015 mg/kg for each APNs. APNs were found to specifically target NP366- and PA224-specific T cells in a dose-dependent manner (FIG. 11), whereas no detectable DiD fluorescence was observed in NP366⁻PA224⁻ T cells. This resulted in a dose-dependent transfection of NP336⁺ or PA224⁺ T cells with the model VHH mRNA (FIG. 5C) compared to minimal transfection (<5%) of NP366⁻ and PA224⁻ CD8⁺ T cells (FIG. 5B). Last, to demonstrate multiplexed transfection with UV-exchanged APNs, a three-plex panel of APNs were synthesized and pooled presenting the top three immunodominant epitopes (NP366, PA224, and GP33) for PR8-GP33 (FIG. 5D). This APN library efficiently transfected the three selective clones of T cells with significantly higher expression of the model VHH protein than T cells in mice treated with PBS (FIG. 5E). Notably, the transfection efficiency across the three T cell clones were comparable with that of PA224/D^{b} APNs administrated alone (FIGS. 4F and 5B), showing that the APN-mediated multiplexed transfection did not compromise the transfection efficiency in each T cell population tested.

Ag-specific CD8⁺ T cells are key players in adaptive immunity, and their ability to directly kill target cells expressing cognate peptide Ags restricted to MHCI presentation is being harnessed for important applications in cell therapy, vaccines, and autoimmunity. Whereas previous work on delivery to T cells via antibodies against cell surface markers (CD3, CD8, etc.) shows great promise, these markers are expressed by all T cells. Moreover, Ag-specific T cell responses are polyclonal; for instance, across five prevalent HLA-A alleles (HLA-A*01:01, HLA-A*02:01, HLA-A*03:01, HLA-A*11:01, and HLA-A*24:02), more than 110 flu-specific peptide epitopes have been identified for human influenza A virus (PR8). Therefore, APNs were developed for multiplexed mRNA delivery to Ag-specific T cells using UV-mediated peptide exchange to expedite production of APNs against a panel of peptide epitopes. The in vivo data using PR8-infected mice showed that APNs with UV-exchanged pMHCI molecules transfected PA224-specific T cells equivalently to APNs synthesized with conventionally folded pMHCI molecules. This allowed construction of a three-plex APN library using UV-mediated peptide exchange to simultaneously transfect the top three immunodominant T cell populations (NP366, PA224, and GP33 specific) in a mouse model of PR8-GP33 flu infection. Notably, while the frequencies of the top three immunodominant T cells in the spleen range from 1% to 4% of the total CD8⁺ T cells, APNs achieved ˜50% transfection efficiency with a model mRNA encoding membrane-anchored VHH. This targeting sensitivity has not been demonstrated before with antibody- and chemical composition-mediated targeting. Moreover, the in vivo transfection efficiency of APNs in T cells was comparatively higher than T cell delivery technologies previously reported, including a PBAE-based polymeric nanoparticle system functionalized with anti-CD3 antibodies (˜10% to 20%) and a lipid-derived polymeric nanoparticle system (˜1.5%). However, note that the mRNA used here was different from the two studies, which used mRNA encoding CAR and Fluc, respectively.

The use of APNs can be expanded for more than three peptide epitopes and beyond the two MHC alleles (H-D^{b} and H-K^{b}) demonstrated in this study. On the basis of prior studies using the UV exchange technology to generate pMHCI libraries with thousands of peptide epitopes, UV exchange can produce an APN library with 10 to 20 viral peptide epitopes per MHC allele, the scale in common viral infection settings (e.g., cytomegalovirus, Epstein-Barr virus, and flu). Moreover, sacrificial UV-labile peptides have been developed for most prevalent HLA alleles, including HLA-A*01:01 (STAPGJLEY) (SEQ ID NO: 13), HLA-A*02:01 (KILGFVFJV) (SEQ ID NO: 14), and HLA-A*11:01 (RVFAJSFIK) (SEQ ID NO: 17). Therefore, APNs are amenable to other pMHCI molecules, including HLA expressed by human CD8+ T cells. The capability of APNs in transfecting multiple virus-specific T cell populations may be used to induce in vivo proliferation of virus-specific T cells to treat virus-mediated cancers. For instance, a fusion protein composed of dimerized pMHCI, and interleukin-2 (IL-2) has been developed to expand HPV16 E7₁₁₋₂₀-specific CD8⁺ T cells to treat HPV-mediated cancers, and a recent study shows that HPV-specific T cells recognizing peptide epitopes derived from HPV E2 and E5 proteins can elicit maxi-mal tumor-reactive CD8⁺ T cell responses against HPV-positive head and neck cancer.

Other than CD8⁺ T cells, the transfection capability of APNs to CD4⁺ T cells can be done by generating APNs with pMHCII. Unlike the cytotoxicity effects triggered by the CD8⁺ TCR-pMHCI interaction, the interaction between CD4⁺ TCR and MHCII induces the differentiation and proliferation of CD4⁺ helperT cells and CD4⁺ T_{reg} cells, which help CD8⁺ T cell responses and suppress pathogenic autoimmunity, respectively. Therefore, prior studies have developed pMHCII-functionalized nanoparticles to expand Ag-specific T_{regs} through pMHC-TCR interaction for the treatment of autoimmune diseases. However, this can be more challenging as binding of class II-bound peptides are less stable compared to that of class I-bound peptides, and the interaction between the pMHCII and CD4⁺ T cells is weaker than that between the class I counterpart and CD8⁺ T cells. Together, these data support the use of APNs for multiplexed mRNA delivery to virus-specific T cells, which can be expanded to transfect broader Ag-specific T cell subsets.

### Materials and Methods

### Animals

Six- to 8-week-old female mice were used at the outsets of all experiments. P14 [B6; D2-Tg(TcrLCMV)327Sdz/JDvsJ], Pmel [B6.Cg-Thy1a/Cy Tg(TcraTcrb)8Rest/J], and OT-1 [C57BL/6-Tg(TcraTcrb)1100Mjb/J]transgenic mice were bred in house using breeding pairs purchased from the Jackson Laboratory. C57BL/6 for PR8 viral infections were purchased from the Jackson Laboratory. All animal procedures were approved by Georgia Tech Institutional Animal Care and Use Committee (protocol numbers: Kwong-A100191, Kwong-A100193, and Santangelo-A100169D).

pMHCI Refolding and Purification

Peptides used for pMHC refolding were synthesized in house using the Liberty Blue Peptide Synthesizer (CEM) and validated using liquid chromatography-mass spectrometry (Agilent). To generate pMHC molecules for bioconjugation, codon-optimized gBlocks for H2-D^{b} and H2-K^{b} b2m were purchased from IDT and cloned into pET3a vectors (Novagen). H2-D^{b} and H2-K^{b} genes were engineered with a C-terminal cysteine by site-directed mutagenesis (New England Biolabs), and pMHC molecules were expressed and refolded as described previously.

### APN Preparation and Characterization

Lipids, including DSPC, cholesterol, DMG-PEG, DSPE-PEG (18:0 PEG2000 PE), and DSPE-PEG2000-maleimide, were purchased from Avanti Polar Lipids. Ionizable lipid D-Lin-MC3-DMA was purchased from MedKoo Biosciences Inc. Fluorescent, lipophilic carbocyanine dye DiD was purchased from Thermo Fisher Scientific. LNP was synthesized as described previously. Briefly, lipid mixture containing MC3, DSPC, cholesterol, DMG-PEG, DSPE-PEG (50:10:38:1.5:0.5 molar ratio), and DiD (1% molar ratio of lipid mix) in ethanol was combined with three volumes of mRNA in acetate buffer [10 mM, pH 4.2, 16:1 (w/w) lipid to mRNA] and injected into microfluidic mixing device NanoAssemblr (PrecisionNanoSystems) at a total flow rate of 12 ml/min (3:1 flow rate ratio aqueous buffer to ethanol). mRNA encoding eGFP, Fluc, and membrane-anchored VHH antibody were gifts from P.J.S. The resultant LNPs were diluted 40× in PBS and concentrated down using Amicon spin filter (10 kDa; Millipore).

To functionalize the synthesized LNPs with pMHC, pMHC was first coupled with DSPE-PEG-maleimide and decorated on LNPs via postinsertion. Briefly, a lipid solution of DSPE-PEG and DSPE-PEG2000-maleimide at 4:1 molar ratio was dried under nitrogen and placed in a vacuum chamber for 2 hours to form a thin film. Lipids were rehydrated in PBS at 6.4 mg/ml in a 60° C. water bath for 15 min and sonicated in an ultrasonic bath (Branson) for 5 min. Refolded pMHCI monomers with C-terminal cysteine were reduced with TCEP (1:3 pMHC to TCEP molar ratio) at 37° C. for 2 hours and mixed with the lipid mixture at room temperature (RT) overnight at 2:1 pMHC/maleimide molar ratio. Lipid-modified pMHCI molecules were incubated with LNPs at 1:50 maleimide/D-Lin-MC3-DMA molar ratio at RT for 6 hours to incorporate pMHCI onto LNPs. The resultant postinsertion mixture was placed in 1 MDa Float-A-Lyzer (Spectrum) and dialyzed against PBS for 16 hours.

The sizes of APNs in PBS were measured by dynamic light scattering with Malvern Nano ZS Zetasizer (Malvern). Final lipid concentration was quantified using a phospholipid assay kit (Sigma-Aldrich). The concentration of conjugated pMHCI was determined using a BCA assay kit (Sigma-Aldrich). The mRNA encapsulation efficiency was quantified by Quant-iT RiboGreen RNA assay (LifeTechnology). Briefly, 50 ml of diluted APNs was incubated with 50 ml of 2% Triton X-100 (Sigma-Aldrich) in TE buffer (10 mM tris-HCl and 20 mM EDTA) in a 96-well fluorescent plate (Costar, Corning) for 10 min at 37° C. to permeabilize the particle. Then, 100 ml of 1% RiboGreen reagent in TE buffer was added into each well, and the fluorescence (excitation wavelength, 485 nm; emission wavelength, 528 nm) was measured using a platereader (BioTek).

### Primary T Cell Isolation and Activation

Spleens isolated from P14, Pmel, or OT-1 TCR transgenic mice were dissociated in complete RPMI media [RPMI 1640 (Gibco)+10% fetal bovine serum (FBS; Gibco)+1% penicillin-streptomycin (Gibco)], and red blood cells were lysed using RBC lysis buffer (BioLegend). CD8+ T cells were isolated using a CD8a+ T cell isolation kit (Miltenyi Biotec). For T cell activation, isolated CD8 T cells were cultured in T cell media [complete RPMI media supplemented with 1× nonessential amino acids (Gibco)+1×10-3 M sodium pyruvate (Gibco) +0.05×10-3 M 2-mercaptoethanol (Sigma-Aldrich)] supplemented with soluble anti-mouse CD28 (5 mg/ml; BD Pharmingen) and rhIL-2 (30 U/ml; Roche) at 1×10⁶ cells/ml in wells coated with anti-mouse CD3e (3 μg/ml; BD Pharmingen).

### In Vitro T Cell Binding and Transfection by APNs

P14, Pmel, and OT-1 CD8⁺ T cell s (1×10⁶ cells per sample) were isolated and incubated with APNs (10 μg/ml) in fluorescence-activated cell sorting (FACS) buffer (1× Dulbecco's PBS+2% FBS+1 mM EDTA+25 mM HEPES) (HEPES buffer: (4-(2-hydroxyethyl)-1-piperazineethanesulfonic acid) for 30 min at 37° C. Cells were washed three times with 1 ml of FACS buffer before analysis on a BD Accuri C6. For validation of in vitro transfection, P14 CD8⁺ T cells were activated for 24 hours as described above and resus-pended in T cell media+rhIL-2 (30 U/ml; Roche) at 2×10⁶ cell/ml. Cells (5×10⁵) were coincubated with GP33/D^{b} APN containing eGFP mRNA (1 mg) in 24-well plates at 37° C. After 4 hours, 700 ml of T cell media+rhIL-2 (30 U/ml; Roche) was added to each well. After an additional 48-hour incubation, cells were washed three times and stained against aCD8 monoclonal antibody (mAb; clone53-6.7, BioLegend; table S2) at 4° C. for 30 min. Cells underwent another two washes with FACS buffer before analysis on BD Accuri C6.

### Quantification of APN Internalization by Acid Wash

OT-1 CD8⁺ T cells were isolated as described above and incubated with OVA/Kb or GP33/D^{b} APNs at 10 mg/ml and aCD8 mAb (clone53-6.7, BioLegend; table S2) at 4° or 37° C. for 30 min. Cells were washed with FACS buffer, and a portion of stained cells was analyzed on a BD Accuri C6. The remaining cells were incubated in an acid wash solution (0.5 M NaCl+0.5 M acetic acid, pH 2.5) for 5 min to strip cell surface proteins as described previously before reanalysis on a BD Accuri C6.

### In Vivo Transfection at Organ Level Using P14 TCR Transgenic Mice

P14 TCR transgenic mice were injected intravenously with GP33/D^{b} or GP100/D^{b} APNs loaded with mRNA encoding Fluc (0.1 mg/kg). Organs were harvested 6 hours after injection and incubated in PBS on ice before IVIS analysis. Organs were soaked in d-luciferin solution (2 mM luciferin) in PBS for 5 min. After 5-min incubation, bio-luminescence images were collected with a Xenogen IVIS Spectrum Imaging System (Xenogen, Alameda, CA). The same type of organs was separated from other organs and imaged together (i.e., spleens from all treatment groups were imaged together).

### In Vivo T Cell Targeting and Transfection in TCR Transgenic Mice

P14 or Pmel TCR transgenic mice were injected intravenously with GP33/D^{b} or GP100/D^{b} APNs loaded with GPI-anchored camelid VHH antibody mRNA (0.2 mg/kg). Splenocytes were harvested 24 hours later and stained against aCD8 mAb (clone 53-6.7, BioLegend), anti-camelid VHH antibody (clone 96A3F5, GenScript), and pMHC tetramers (streptavidin, 2 mg/ml) on ice for 30 min. The working concentrations of antibodies were listed in table S2. Epitope pMHC tetramers for staining were synthesized in house by mixing biotinylated pMHC with fluorescently labeled streptavidin at a 4:1 molar ratio. Cells were washed three times with FACS buffer before analysis on BD Accuri C6 All flow data in this study were analyzed with FlowJo v.10 (Tree Star).

### In Vivo T Cell Targeting and Transfection in PR8-Infected Mice

PR8 virus was a gift from P.J.S. PR8-GP33 was a gift from R.A. (Emory University) and E. J. Wherry (University of Pennsylvania). Six- to 8-week-old PR8-infected C57BL/6 mice were intranasally infected with either PR8 virus or PR8-GP33 recombinant virus, as specified in Results and figure captions. PR8-infected mice were injected intravenously with NP366/D^{b} and PA224/D^{b} APNs containing the GPI-anchored camelid VHH antibody mRNA (0.03 or 0.2 mg/kg) on day 10 after viral infection. Twenty-four hours after the injection, splenocytes were harvested as described above for immunofluorescent staining. Cells were stained against tetramers (NP366/D^{b}, PA224/D^{b}, 0.2 mg of streptavidin/staining sample), aCD8a mAb (clone 53-6.7, BD), aNK1.1 mAb (clone PK136, Tonbo), aB220 mAb (clone RA3-6B2, Tonbo), aCD4 mAb (cloneRM4-2, Tonbo), and anti-camelid VHH antibody (clone 96A3F5, GenScript) on ice for 30 min. Antibodies were all used at 1:100 dilutions, and the specific working concentrations were listed in table S2. Cells were then fixed with IC fixation buffer (Thermo Fisher Scientific) for the flow analysis (Fortessa, BD).

### Functional Distribution of APNs at Cellular Level

Six- to 8-week-old PR8-infected C57BL/6 mice were injected intra-venously with conventional or UV-exchanged PA224/D^{b} APNs containing the GPI-anchored camelid VHH antibody mRNA (0.1 mg/kg) on day 10 after viral infection. Twenty-four hours after the injection, cells from spleen and liver were harvested as described above. Cells were stained against aCD8a mAb (clone 53-6.7, BD), aNK1.1 mAb (clone PK136, Tonbo), aB220 mAb (clone RA3-6B2, Tonbo), aCD31 mAb (clone PK136, Tonbo), aCD45 mAb (clone 30-F11, BioLegend), aCD4 mAb (clone RM4-2, Tonbo), aCD11b mAb(clone M1/70, BioLegend), aCD11c mAb (clone N418, BioLegend), aLy6c mAb (clone HK1.4, BioLegend), aF4/80 mAb (clone BM8, BioLegend), and anti-camelid VHH antibody (clone 96A3F5, GenScript) on ice for 30 min. Antibodies were all used at 1:100 dilutions, and the specific working concentrations were listed in table S2. Cells were then fixed with IC fixation buffer (Thermo Fisher Scientific) for the flow analysis (Fortessa, BD). Cells were identified by a combination of surface markers: macrophages (CD45⁺, CD11b⁺, CD11c⁻, and Ly6c⁻/low), dendritic cells (CD45⁺, CD11c⁺, and CD11b⁻), endothelial cells (CD45⁻ and CD31⁺), monocytes (CD45⁺ CD11b⁺, CD11c⁻, and Ly6c⁺), B cells (CD45⁺ and B220⁺), CD4⁺ T cells (CD45⁺ and CD4⁺), CD8⁺ T cells (CD45⁺, CD8⁺, and NK1.1⁻), flu-specific CD8⁺ T cells (CD45⁺, CD8⁺, NK1.1⁻, and tet⁺), NK cells (CD45⁺ and NK1.1⁺), hepatocytes (CD31⁻, CD45⁻, and F4/80⁻), and Kupffer cells (CD31⁻, CD45⁺, and F4/80⁺).

### Statistical Analysis

Significant differences between control and treatment groups were determined by various statistical analyses. Student's t test was used for two-group comparison. One-way analysis of variance (ANOVA) was used for multiple-group comparison. Two-way ANOVA was used when there were subgroups in each group. Data represent means±SD in each figure and table as indicated. Statistical analyses were performed using GraphPad Prism 8.0.2 software (GraphPad Software) (*P<0.05, **P<0.005, ***P<0.0005, and ****P<0.0001).

TABLE 1
Sacrificial peptides for UV exchange in class
I pMHC alleles

| MHC alleles | Sacrificial peptides |
|---|---|
| H2-D^{b} | ASNENJETM (SEQ ID NO: 10) |
| H2-K^{b} | FAPGNYJAL (SEQ ID NO: 11) |
| H2-L^{b} | YPNVNIHJF (SEQ ID NO: 12) |
| HLA-A*01:01 | STAPGJLEY (SEQ ID NO: 13) |
| HLA-A*02:01 | KILGFVFJV (SEQ ID NO: 14) |
| HLA-A*03:01 | RIYRJGATR (SEQ ID NO: 15) |
| HLA-A*07:02 | AARGJTLAM (SEQ ID NO: 16) |
| HLA-A*11:01 | RVFAJSFIK (SEQ ID NO: 17) |

J: UV-sensitive amino acid (3-Amino-3-(2-nitro)phenyl-propionic acid). Reference: Toebes et al., Curr Protoc Immunol. 2009.

Modulatory molecules that can be delivered using antigen-presenting nanoparticles (APNs) include but not limit to: CRISPR/Cas, siRNA, (inducible) caspase, Foxp3 transcription factor, transforming growth factor beta (TGF-beta). The modulatory molecules can be delivered in the form of mRNA, mRNA, plasmid DNA, mini-circle DNA, or proteins.

Protease-mediated peptide exchange on pMHC class 11 molecules: In addition to the UV-mediated peptide exchange protocol for pMHC class I APNs, a previously reported protocol for pMHC class II (pMHCII) can be implemented to APNs. Briefly, the pMHC class II recombinant proteins are expressed using a derivative cell line of 293T. The pMHCII molecules are engineered to carry a sacrificial peptide that is linked to the N-terminus of beta chain through a protease cleavable peptide linker. The C-terminus of alpha chain can be engineered with a Cys to allow for lipid conjugation to make APNs. The pMHCII molecules are first conjugated to a lipid through thiol-maleimide chemistry. The sacrificial peptide is subsequently exchanged into the peptide of interest in the presence of HRV-3C protease, HLA-DM, and the peptide of interest. The exchanged pMHCII molecules are then modified to the APN core through post-insertion.

TABLE 2
Autoimmune diseases treatment using APNs to modulate
T cells as prophylactic or therapeutic measures

| Disease | T cells | HLA alleles |
|---|---|---|
| Multiple sclerosis | CD8+ and CD4+ | HLA-DRB2 (II) |
| Type 1 diabetes | CD8+ and CD4+ | HLA-A, B (I), HLA-DQ2/DQ8, |
| | | DQ4, DQ6, DR3 (II), and more. |
| Celiac disease | CD4+ | HLA-DQ2.5/DQ8 (II) |
| Primary biliary | CD8+ and CD4+ | HLA-DRB1, DQB1 (II) |
| cholangitis | | |
| Pemphigus | CD8+ and CD4+ | HLA-DR4 (II) |
| vulgaris | | |
| Goodpasture | CD4+ | HLA-DR15, HLA-DR2 (II) |
| syndrome | | |
| Grave's disease | CD4+ | HLA-B (I), HLA-DR3, DRB1(II) |
| Autoimmune uveitis | CD8+ and CD4+ | HLA-B27 (I), HLA-DQ1 (II) |
| Rheumatoid arthritis | CD4+ | HLA-DR4, DRB1 (II) |
| Inflammatory bowel disease | CD4+ | HLA-C (I), HLA-DRB1 (II) |
| Systemic lupus erythematosus | CD4+ | HLA-DR2, DR3 (II) |

TABLE 3
Proteins related to autoimmune diseases:

| Disease | T cells | HLA alleles | Proteins |
|---|---|---|---|
| Multiple sclerosis | CD8+ and | HLA-DRB2 (II) | Myelin |
| | CD4+ | | |
| Type 1 diabetes | CD8+ and | HLA-A, B (I), HLA- | Insulin, GAD65/67, IGRP, |
| | CD4+ | DQ2/DQ8, DQ4, DQ6, | chromogranin, etc. |
| | | DR3 (II), and more. | |
| Celiac disease | CD4+ | HLA-DQ2.5/DQ8 (II) | Gluten |
| Primary biliary | CD8+ and | HLA-DRB1, DQB1 (II) | Mitochondrial proteins |
| cholangitis | CD4+ | | |
| Pemphigus vulgaris | CD8+ and | HLA-DR4 (II) | Desmosomal glycoprotein |
| | CD4+ | | desmoglein 3 (DSG3) |
| Goodpasture | CD4+ | HLA-DR15, HLA-DR2 | Alpha 3 NC1 |
| syndrome | | (II) | |
| Grave's disease | CD4+ | HLA-B (I), HLA-DR3, | Thyroid-stimulating |
| | | DRB1(II) | hormone receptor (TSHR) |
| Autoimmune | CD8+ and | HLA-B27 (I), HLA-DQ1 | Interphotoreceptor retinoid- |
| uveitis | CD4+ | (II) | binding protein (IRBP) |
| Rheumatoid | CD4+ | HLA-DR4, DRB1 (II) | Galanthus nivalis agglutinin |
| arthritis | | | (GNA), filamin A (FLNA), |
| | | | other citrullinated proteins |
| Inflammatory | CD4+ | HLA-C (I), HLA-DRB1 | N/A |
| bowel disease | | (II) | |
| Systemic lupus | CD4+ | HLA-DR2, DR3 (II) | Nuclear proteins like |
| erythematosus | | | histones |

TABLE 4
Quantification of pMHC on APNs using BCA assay kit.

| APN | | UV | UV |
|---|---|---|---|
| formulation | Bare NP | PA224/D^{b} APN | GP33/D^{b} APN |
| pMHC | 13.21 | 297.97 | 265.74 |
| concentration | | | |
| (μg/mL) | | | |
| pMHC | 0.033 | 0.745 | 0.664 |
| (μg/μg lipids) | | | |

TABLE 5
List of staining reagents for flow cytometry analysis.

| | | | | | |
|---|---|---|---|---|---|
| | | | | | Working |
| Target | Fluorochrome | Clone | Source | Identifier | concentration |

REAGENT
Antibodies

| | | | | | | |
|---|---|---|---|---|---|---|
| Camelid VHH | iFluor488 | 96A3F5 | Genscript | A01862 | 5 | μg/mL |
| CD8 | APC | 53-6.7 | Biolegend | 100712 | 2 | μg/mL |
| CD8 | PE | 53-6.7 | BD | 561095 | 2 | μg/mL |
| CD4 | PerCP/Cy5.5 | RM4-2 | TONBO | 65-0042-U100 | 2 | μg/mL |
| NK1.1 | PerCP/Cy5.5 | PK136 | TONBO | 65-5941-U100 | 2 | μg/mL |
| B220 | PerCP/Cy5.5 | RA3-6B2 | TONBO | 65-0452-U100 | 2 | μg/mL |
| CD31 | PerCP/Cy5.5 | MEC13.3 | Biolegend | 102522 | 2 | μg/mL |
| CD45 | PE/Cy7 | 30-F11 | Biolegend | 103114 | 2 | μg/mL |
| CD11b | BV421 | M1/70 | Biolegend | 101235 | 2 | μg/mL |
| CD11c | BV605 | N418 | Biolegend | 117333 | 2 | μg/mL |
| Ly6c | BV785 | HK1.4 | Biolegend | 128041 | 2 | μg/mL |
| F4/80 | BV421 | BM8 | Biolegend | 123131 | 2 | μg/mL |

Other reagents

| | | | | | |
|---|---|---|---|---|---|
| Streptavidin | PE | Invitrogen | S866 | 10 | μg/mL |
| Streptavidin | APC | Invitrogen | S868 | 6.66 | μg/mL |
| Streptavidin | BV421 | Biolegend | 405225 | 2 | μg/mL |
| LIVE/DEAD ™ | Fixable Aqua | Invitrogen | L34957 | | |
| Stain Kit | Dead Cell | | | | |

### Example 2: Antigen-Specific Targeting to Mouse and Human T Cells Ex Vivo

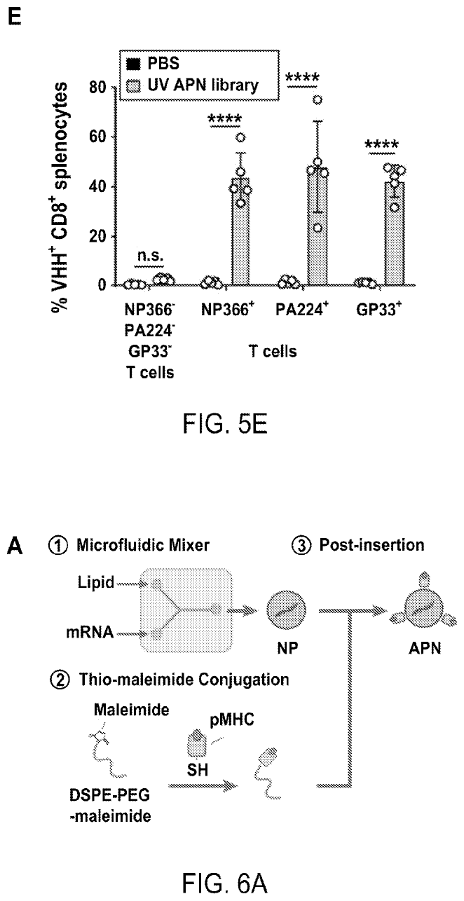

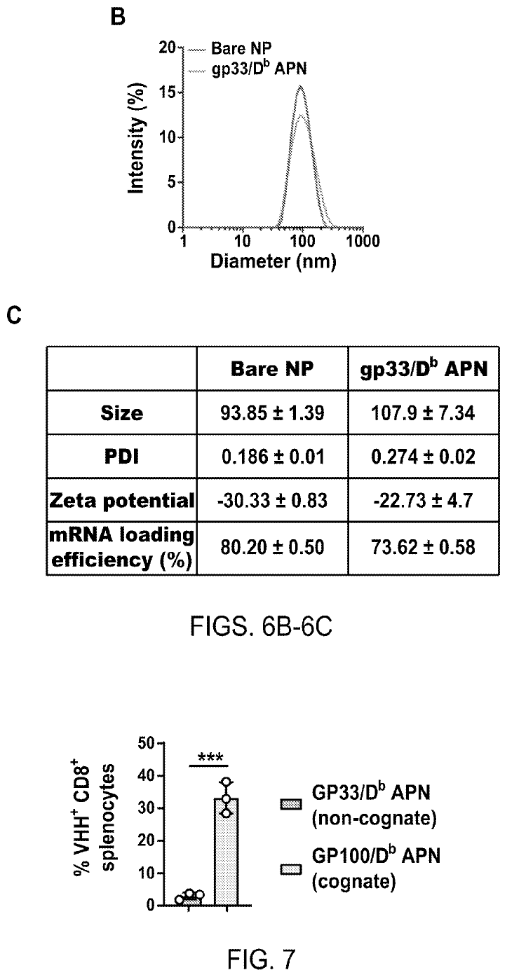

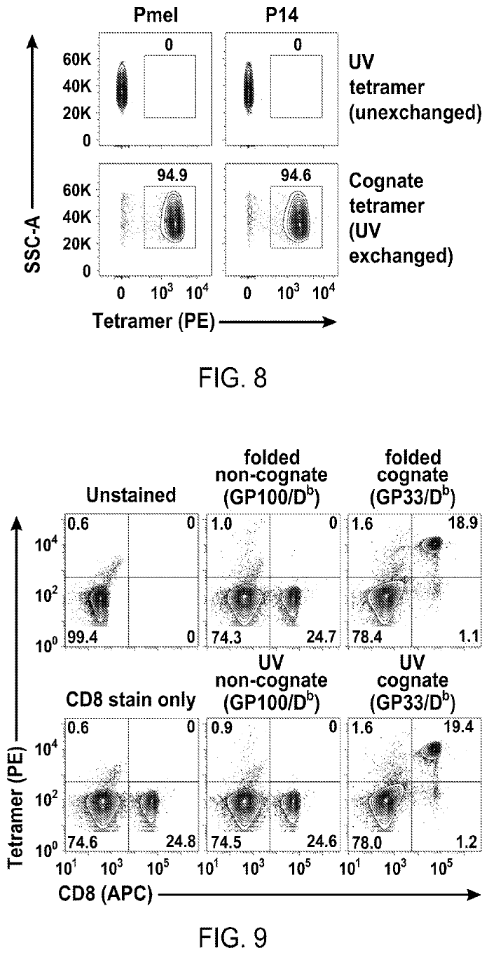

The nanoparticle matrix of the exemplary method includes, but is not limited to, liposomes, lipid nanoparticles, and polymeric nanoparticles. Surfaces of the nanoparticles are functionalized with MHC carrying peptide antigens of interest, and the resulting product is abbreviated as pMHC-NPs henceforth. The method described herein has been demonstrated in MHC class 1 for CD8+ T cell modulation, but it can broadly use for both class 1 (target CD8+ T cells) and class 2 MHC (target CD4+ T cells). To achieve this surface pMHC conjugation in a selective manner, the C-terminal of the heavy chain, a major component of pMHC, was engineered with a cysteine to allow for selective conjugations based on thiol-maleimide chemistry. This bioconjugation approach is applicable to both murine and human MHC by engineering heavy chains of murine (SED ID: 18 & 19) and human (SED ID: 20) pMHC with C-terminal cysteine. Lipid nanoparticles (LNPs) functionalized with MHC carrying the gp33 peptide antigen was found to selectively bind to the matched T cell receptor (TCR) expressed by the CD8+ T cells from P14 mice (FIG. 12). This pMHCTCR interaction induces intracellular cargo delivery to p14 CD8+ T cells (FIG. 13). LNPs functionalized with human pMHC (a.k.a. human leukocyte antigen, HLA) carrying cytomegalovirus (CMV) peptide antigens specifically targeted CMV-specific CD8+ T cells isolated from human blood in the presence of other untargeted CD8+ T cells (FIG. 14).

### Example 3. High Throughput Ligand Exchange by UV Light to Control Targeting Specificity

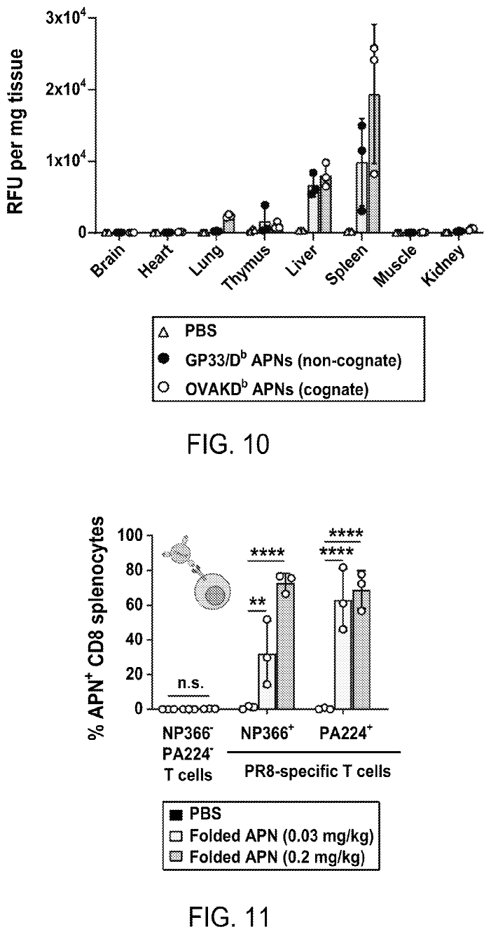

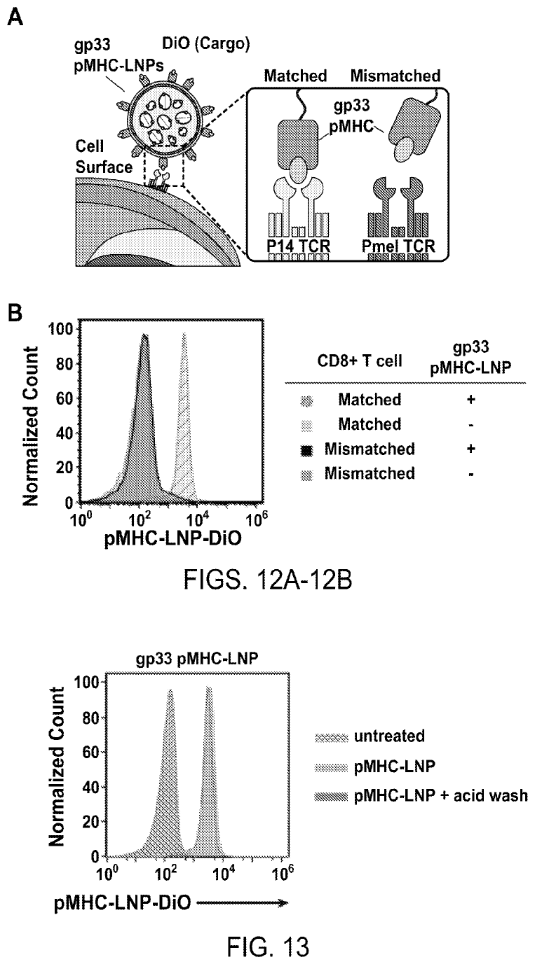

Conventional approaches for ligand conjugation in the nanomedicine field is done with individual conjugation of specific ligands on the nanoparticle surfaces. In the case of pMHC conjugation, the throughout is limited not only by the individual conjugation of MHC loaded with various peptide epitopes to the surface of nanoparticles, but also by the in vitro refolding reactions with specific peptides. This approach is therefore not amenable to high-throughput production of vast collections of MHCs to target a pool of T cells specific for a particular disease setting. To circumvent these challenges, the exemplary method was developed so that it can allow for pMHC conjugation in a high throughput manner (FIG. 15). The MHCs were refolded with sacrificial peptides (e.g., FAPGNYXAL (SEQ ID NO: 28) for murine H2-Db) that are derived from high affinity epitopes modified with an unnatural UV-labile amino acid (where X is the UV-labile residue) at one of the anchor residues which is crucial for peptide binding. This UV-cleavable pMHC was conjugated on the surface of preformed lipid micelles through thiol-maleimide chemistry. Subsequently, UV exchange was performed in the presence of peptides of interests to produce pMHCs loaded with the desired peptide (e.g., gp33). The pMHC micelles were then incubated with LNPs for 6 hrs to allow surface modification of pMHC on LNPS. The resulting pMHC-LNPs demonstrated comparable size distribution with pMHCLNPs generated with MHC initially refolded with gp33. Moreover, comparable staining has been observed using the exchanged pMHC-LNPs compared to the conventional tetramers or pMHC-LNPs generated with MHC initially refolded with gp33 (FIGS. 16 & 17). The production of the UV-cleavable pMHC molecules by the standard in vitro refolding reaction can be achieved within 2 weeks, and the actual high-throughput MHC peptide exchange and subsequent nanoparticle conjugations can be done in less than a day. The exemplary method therefore can accelerate the synthesis of pMHC-NPs with a collection of T-cell targeting specificity, while reducing the required labor and resources.

### Example 4. Programing Antigen-Specific T Cells In Vitro and In Vivo

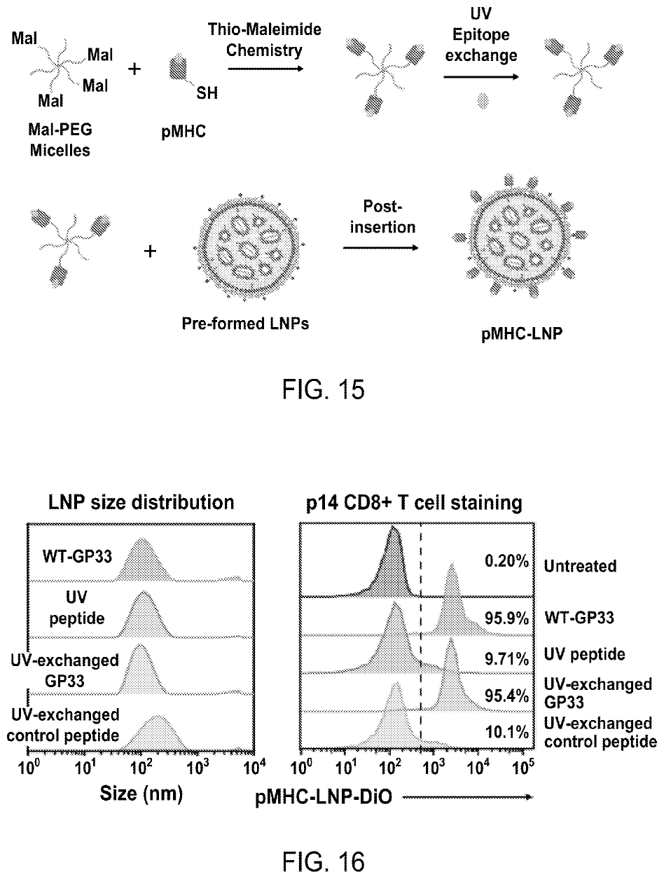

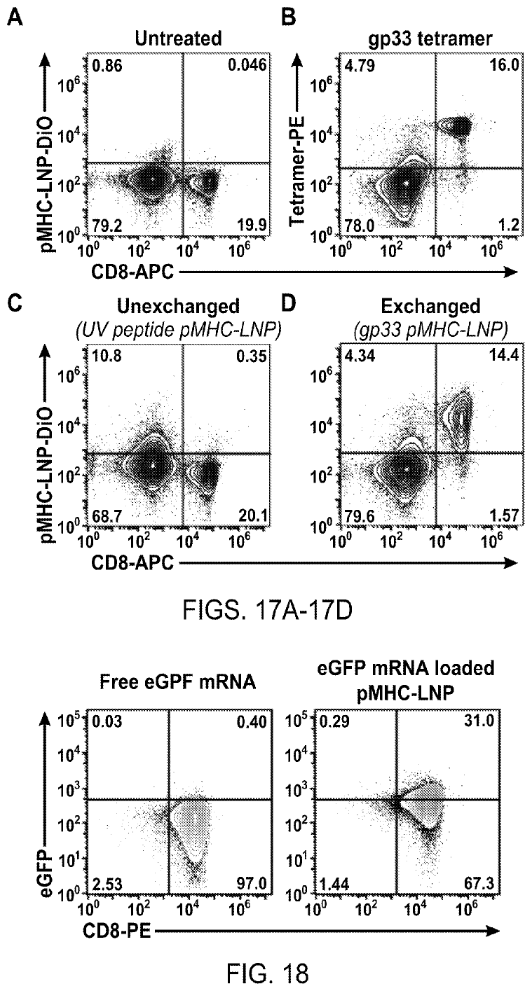

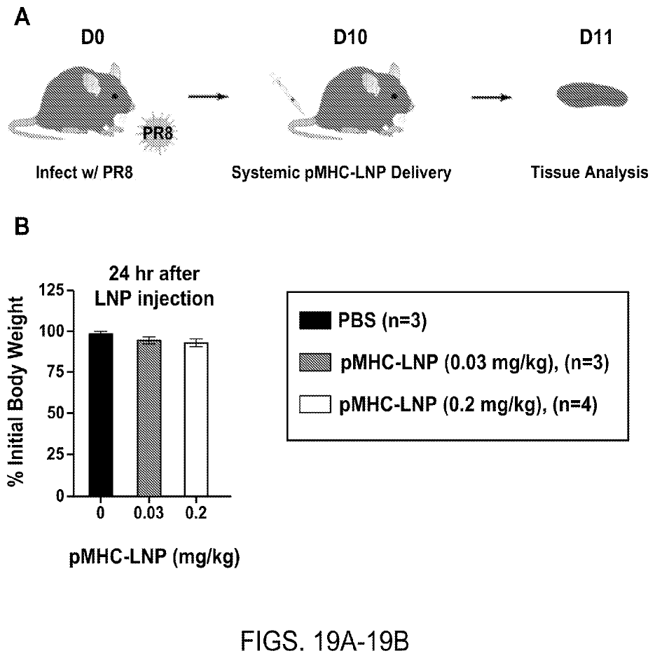

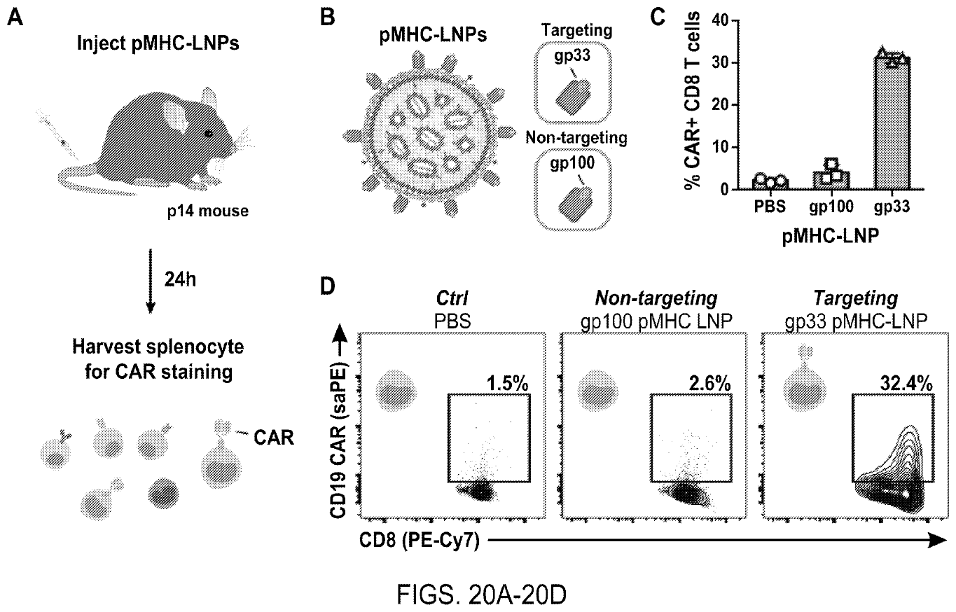

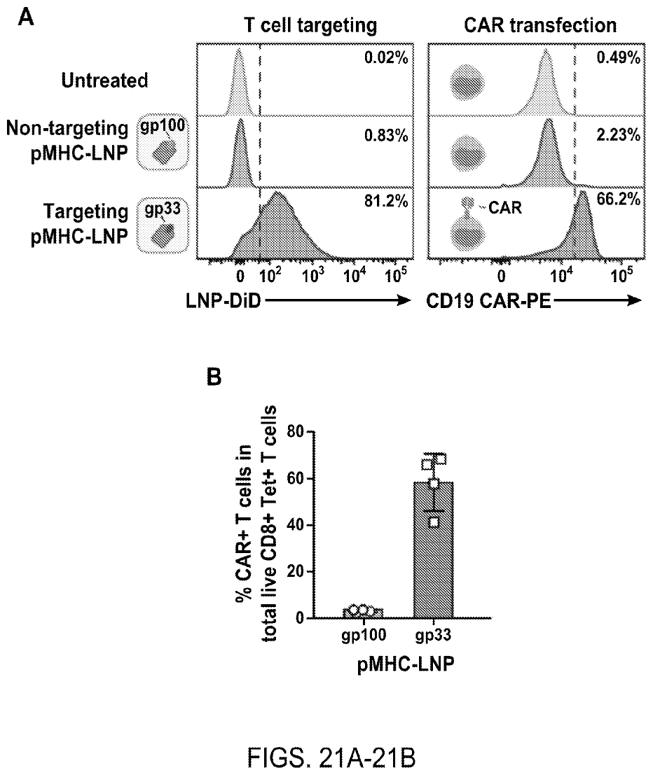

To program T cells with the pMHC-NPs, cargos that can be loaded in the pMHC-NPs include but not limit to mRNA, plasmid DNA, mini-circle DNA, CRISPR/Cas9, proteins, and small molecules. pMHC-NPs loaded with a model eGFP mRNA achieved 31% of transfection efficiency in P14 CD8+ T cells in vitro, whereas detectable transfection was observed from free mRNA (<1%) (FIG. 18). In a murine model recapitulating human influenza infections (PR8, H1N1) (FIG. 19A), no significant body weight decrease was observed in mice treated with pMHC-NPs at a mRNA dose of 0.03 and 0.2 mg/kg (FIG. 19B), indicating that pMHC-NPs did not induce acute toxicity. Moreover, pMHC-NPs specifically targeted immunodominant flu-specific T cells (i.e., T cells recognizing NP366 and PA224 flu epitopes) in a dose-dependent manner (FIG. 19C), whereas no detectable targeting of pMHC-NPs was observed in rest of 106-108 different T cell clones (NP366−PA224−). This antigen-specific targeting of pMHC-LNPs also resulted in a dose-dependent transfection of T cells with a model mRNA encoding GPI-anchored camelid nanobody (FIG. 19D). Moreover, pMHC-NPs were capable of carrying mRNA encoding clinical grade CAR against either mouse CD19 (mCD19) or human CD19 (hCD19) (SEQ ID NOs: 26 & 27, FIG. 20-22). With the workflow showing in FIG. 20a, LNPs functionalized with targeting gp33 pMHC transfected 30% of CD8+ T cells with hCD19 CAR mRNA in p14 mice, which was significantly higher than the counterpart modified with non-targeting gp100 pMHC (FIGS. 20B-20D). In addition to hCD19 CAR, the pMHC-LNPs can also target and transfect CD8+ T cells in p14 mice with mCD19 CAR mRNA in an antigen-specific manner (FIG. 21). As shown in FIG. 21, pMHC-LNPs targeted >80% of CD8+ T cells, which resulted in ˜60% transfection efficiency of mCD19 CAR mRNA in vivo. In comparison to co-incubation with wild type EL4 cancer cells, T cells transfected by targeting gp33 pMHC-LNPs led to higher cytotoxicity when co-incubated with EL4 expressing the target mCD19 antigens (FIGS. 22A-22C). Furthermore, only background cytotoxicity signals were observed in from the T cells in vivo transfected with non-targeting gp100 pMHCLNPs. In accordance with these results, T cells transfected by targeting gp33 pMHC-LNPs led to significant higher IFN-γ secretion than the counterparts transfected by non-targeting gp100 pMHC-LNPs, when cocultured with target EL4-mCD19 cells (FIG. 22D).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

### Sequences

SEQ ID NO: 18: Murine MHC class I heavy chain H2-Db engineered with C-terminal cysteine
ATGGGCCCACACTCGATGCGGTATTTCGAGACCGCCGTGTCCCGGCCCGGCC
TCGAGGAGCCCCGGTACATCTCTGTCGGCTATGTGGACAACAAGGAGTTCGTGCGCT
TCGACAGCGACGCGGAGAATCCGAGATATGAGCCGCGGGCGCCGTGGATGGAGCA
GGAGGGGCCGGAGTATTGGGAGCGGGAAACACAGAAAGCCAAGGGCCAAGAGCAG
TGGTTCCGAGTGAGCCTGAGGAACCTGCTCGGCTACTACAACCAGAGCGCGGGCGG
CTCTCACACACTCCAGCAGATGTCTGGCTGTGACTTGGGGTCGGACTGGCGCCTCCT
CCGCGGGTACCTGCAGTTCGCCTATGAAGGCCGCGATTACATCGCCCTGAACGAAG
ACCTGAAAACGTGGACGGCGGCGGACATGGCGGCGCAGATCACCCGACGCAAGTG
GGAGCAGAGTGGTGCTGCAGAGCATTACAAGGCCTACCTGGAGGGCGAGTGCGTGG
AGTGGCTCCACAGATACCTGAAGAACGGGAACGCGACGCTGCTGCGCACAGATTCC
CCAAAGGCACATGTGACCCATCACCCCAGATCTAAAGGTGAAGTCACCCTGAGGTG
CTGGGCCCTGGGCTTCTACCCTGCTGACATCACCCTGACCTGGCAGTTGAATGGGGA
GGAGCTGACCCAGAAAATGGAGCTTGTGGAGACCAGGCCTGCAGGGGATGGAACCT
TCCAGAAGTGGGCATCTGTGGTGGTGCCTCTTGGGAAGGAGCAGAATTACACATGC
CGTGTGTACCATGAGGGGCTGCCTGAGCCCCTCACCCTGAGATGGGAGCCTCCTCCA
TCCACTGGCAGCGGTGGTTCCGGTGGTTCCGCGTGT
SEQ ID NO: 19: Murine MHC class I heavy chain Kb engineered with C-terminal cysteine
ATGGGTCCACACTCTCTGCGCTATTTCGTTACGGCTGTTAGCCGTCCGGGTCT
GGGTGAGCCGCGCTACATGGAAGTCGGTTACGTCGACGACACCGAATTCGTGCGTT
TCGACAGCGACGCGGAGAACCCGCGTTATGAGCCGCGTGCGCGTTGGATGGAGCAG
GAAGGTCCGGAGTACTGGGAGCGTGAAACGCAAAAGGCGAAGGGCAATGAACAGA
GCTTTCGTGTTGATCTGCGCACTCTGCTGGGTTACTACAACCAGAGCAAAGGTGGCA
GCCATACCATTCAGGTGATTAGCGGTTGTGAAGTCGGCTCTGATGGCCGCCTGTTGC
GCGGTTATCAGCAATATGCATACGACGGTTGCGACTACATTGCGCTGAATGAAGAT
CTGAAAACGTGGACTGCGGCGGACATGGCCGCACTGATTACCAAACACAAGTGGGA
GCAAGCGGGCGAAGCCGAGCGCCTGCGTGCGTATCTGGAAGGCACCTGTGTGGAAT
GGCTGCGCCGCTATCTGAAGAATGGCAATGCCACGTTGCTGCGTACGGATTCCCCGA
AAGCGCACGTGACGCACCATAGCCGTCCTGAGGATAAAGTTACCCTGCGTTGCTGG
GCACTGGGCTTTTACCCGGCAGATATCACCTTGACGTGGCAACTGAATGGTGAAGA
GCTGATTCAGGATATGGAACTGGTGGAGACTCGTCCGGCTGGCGACGGTACCTTCC
AGAAATGGGCATCGGTTGTCGTCCCTCTGGGTAAAGAGCAATACTATACCTGCCAC
GTTTACCACCAAGGTCTGCCGGAGCCGCTGACCTTGCGTTGGGAGCCACCGCCGAG
CACCGGCAGCGGTGGTAGCGGCGGTTCCGCGTGT
SEQ ID NO: 20: Human HLA-A0201 class I heavy chain engineered with C-terminal cysteine
ATGGGTTCTCATTCTATGAGATATTTCTTCACATCCGTGTCCCGGCCCGGCCG
CGGGGAGCCCCGCTTCATCGCAGTGGGCTACGTGGACGACACGCAGTTCGTGCGGT
TCGACAGCGACGCCGCGAGCCAGAGGATGGAGCCGCGGGCGCCGTGGATAGAGCA
GGAGGGTCCGGAGTATTGGGACGGGGAGACACGGAAAGTGAAGGCCCACTCACAG
ACTCACCGAGTGGACCTGGGGACCCTGCGCGGCTACTACAACCAGAGCGAGGCCGG
TTCTCACACCGTCCAGAGGATGTATGGCTGCGACGTGGGGTCGGACTGGCGCTTCCT
CCGCGGGTACCACCAGTACGCCTACGACGGCAAGGATTACATCGCCCTGAAAGAGG
ACCTGCGCTCTTGGACCGCGGCGGACATGGCAGCTCAGACCACCAAGCACAAGTGG
GAGGCGGCCCATGTGGCGGAGCAGTTGAGAGCCTACCTGGAGGGCACGTGCGTGGA
GTGGCTCCGCAGATACCTGGAGAACGGGAAGGAGACGCTGCAGCGCACGGACGCC
CCCAAAACGCATATGACTCACCACGCTGTCTCTGACCATGAAGCCACCCTGAGGTGC
TGGGCCCTGAGCTTCTACCCTGCGGAGATCACACTGACCTGGCAGCGGGATGGGGA
GGACCAGACCCAGGACACGGAGCTCGTGGAGACCAGGCCTGCAGGGGATGGAACC
TTCCAGAAGTGGGCGGCTGTGGTGGTGCCTTCTGGACAGGAGCAGAGATACACCTG
CCATGTGCAGCATGAGGGTTTGCCCAAGCCCCTCACCCTGAGATGGGAGCCGGCTA
GCTGC
SEQ ID NO: 21: Human beta-2 microglobulin
ATCCAGCGTACCCCGAAGATTCAGGTGTACTCACGCCATCCAGCTGAGAACG
GCAAAAGTAACTTTCTGAATTGCTATGTCTCGGGCTTCCACCCGTCCGATATCGAAG
TGGACCTTCTGAAAAACGGTGAACGGATCGAAAAAGTCGAGCATTCTGACTTATCA
TTCAGCAAAGATTGGTCTTTTTACTTGTTATATTATACCGAATTTACCCCGACAGAA
AAAGATGAGTATGCATGCCGTGTCAACCACGTTACGCTGTCCCAGCCGAAAATCGT
GAAATGGGATCGTGATATGTAA
SEQ ID NO: 22: Murine MHC class II I-Ag7 alpha chain engineered with C-terminal cysteine
ATGCCGCGCAGCAGAGCTCTGATTCTGGGGGTCCTCGCCCTGAACACCATGC
TCAGCCTCTGCGGAGGTGAAGACGACATTGAGGCCGACCACGTAGGCTTCTATGGT
ACAACTGTTTATCAGTCTCCTGGAGACATTGGCCAGTACACACATGAATTTGATGGT
GATGAGTTGTTCTATGTGGACTTGGATAAGAAGAAAACTGTCTGGAGGCTTCCTGAG
TTTGGCCAATTGATACTCTTTGAGCCCCAAGGTGGACTGCAAAACATAGCTGCAGAA
AAACACAACTTGGGAATCTTGACTAAGAGGTCAAATTTCACCCCAGCTACCAATGA
GGCTCCTCAAGCGACTGTGTTCCCCAAGTCCCCTGTGCTGCTGGGTCAGCCCAACAC
CCTTATCTGCTTTGTGGACAACATCTTCCCACCTGTGATCAACATCACATGGCTCAG
AAATAGCAAGTCAGTCACAGACGGCGTTTATGAGACCAGCTTCCTCGTCAACCGTG
ACCATTCCTTCCACAAGCTGTCTTATCTCACCTTCATCCCTTCTGATGATGACATTTA
TGACTGCAAGGTGGAGCACTGGGGCCTGGAGGAGCCGGTTCTGAAACACTGGGAAC
CTGAGATTCCAGCCCCCATGTCAGAGCTGACAGAGGTCGACACTACAGCTCCATCA
GCTCAGTTGAAAAAGAAATTGCAAGCACTGAAGAAAAAGAACGCTCAGCTGAAGT
GGAAACTTCAAGCCCTCAAGAAGAAACTCGCCCAGTCCGGGAGCGGATCTGGCTGT
SEQ ID NO: 23: Murine MHC class II I-Ag7 beta chain
GGAGACTCCGAAAGGCATTTCGTGCACCAGTTCAAGGGCGAGTGCTACTTCA
CCAACGGGACGCAGCGCATACGGCTCGTGACCAGATACATCTACAACCGGGAGGAG
TACCTGCGCTTCGACAGCGACGTGGGCGAGTACCGCGCGGTGACCGAGCTGGGGCG
GCACTCAGCCGAGTACTACAATAAGCAGTACCTGGAGCGAACGCGGGCCGAGCTGG
ACACGGCGTGCAGACACAACTACGAGGAGACGGAGGTCCCCACCTCCCTGCGGCGG
CTTGAACAGCCCAATGTCGCCATCTCCCTGTCCAGGACAGAGGCCCTCAACCACCAC
AACACTCTGGTCTGTTCGGTGACAGATTTCTACCCAGCCAAGATCAAAGTGCGCTGG
TTCAGGAATGGCCAGGAGGAGACAGTGGGGGTaTCATCCACACAGCTTATTAGGAA
TGGGGACTGGACCTTCCAGGTCCTGGTCATGCTGGAGATGACCCCTCATCAGGGAG
AGGTCTACACCTGCCATGTGGAGCATCCCAGCCTGAAGAGCCCCATCACTGTGGAG
TGGAGGGCACAGTCCGAGTCTGCCCGGAGCAAGGGATCCACTACAGCTCCATCAGC
TCAGCTCGAAAAAGAGCTCCAGGCCCTGGAGAAGGAAAATGCACAGCTGGAATGG
GAGTTGCAAGCACTGGAAAAGGAACTGGCTCAG
SEQ ID NO: 24: Murine MHC class II I-Ab alpha chain engineered with C-terminal cysteine
ATGCCGCGCAGCAGAGCTCTGATTCTGGGGGTCCTCGCCCTGACCACCATGC
TCAGCCTCTGTGGAGGTGAAGACGACATTGAGGCCGACCACGTAGGCACCTATGGT
ATAAGTGTATATCAGTCTCCTGGAGACATTGGCCAGTACACATTTGAATTTGATGGT
GATGAGTTGTTCTATGTGGACTTGGATAAGAAGGAGACTGTCTGGATGCTTCCTGAG
TTTGGCCAATTGGCAAGCTTTGACCCCCAAGGTGGACTGCAAAACATAGCTGTAGTA
AAACACAACTTGGGAGTCTTGACTAAGAGGTCAAATTCCACCCCAGCTACCAATGA
GGCTCCTCAAGCGACTGTGTTCCCCAAGTCCCCTGTGCTGCTGGGTCAGCCCAACAC
CCTCATCTGCTTTGTGGACAACATCTTCCCTCCTGTGATCAACATCACATGGCTCAG
AAATAGCAAGTCAGTCGCAGACGGTGTTTATGAGACCAGCTTCTTCGTCAACCGTGA
CTATTCCTTCCACAAGCTGTCTTATCTCACCTTCATCCCTTCTGACGATGACATTTAT
GACTGCAAGGTGGAACACTGGGGCCTGGAGGAGCCGGTTCTGAAACACTGGGAACC
TGAGATTCCAGCCCCCATGTCAGAGCTGACAGAGGTCGACACTACAGCTCCATCAG
CTCAGTTGAAAAAGAAATTGCAAGCACTGAAGAAAAAGAACGCTCAGCTGAAGTG
GAAACTTCAAGCCCTCAAGAAGAAACTCGCCCAGTCCGGGAGCGGATCTGGCTGT
SEQ ID NO: 25: Murine MHC class II I-Ab beta chain
GGAGACTCCGAAAGGCATTTCGTGTACCAGTTCATGGGCGAGTGCTACTTCA
CCAACGGGACGCAGCGCATACGATATGTGACCAGATACATCTACAACCGGGAGGAG
TACGTGCGCTACGACAGCGACGTGGGCGAGCACCGCGCGGTGACCGAGCTGGGGCG
GCCAGACGCCGAGTACTGGAACAGCCAGCCGGAGATCCTGGAGCGAACGCGGGCC
GAGCTGGACACGGTGTGCAGACACAACTACGAGGGGCCGGAGACCCACACCTCCCT
GCGGCGGCTTGAACAGCCCAATGTCGTCATCTCCCTGTCCAGGACAGAGGCCCTCA
ACCACCACAACACTCTGGTCTGCTCAGTGACAGATTTCTACCCAGCCAAGATCAAAG
TGCGCTGGTTCCGGAATGGCCAGGAGGAGACGGTGGGGGTCTCATCCACACAGCTT
ATTAGGAATGGGGACTGGACCTTCCAGGTCCTGGTCATGCTGGAGATGACCCCTCG
GCGGGGAGAGGTCTACACCTGTCACGTGGAGCATCCCAGCCTGAAGAGCCCCATCA
CTGTGGAGTGGAGGGCACAGTCTGAGTCTGCCCGGAGCAAGGGATCCACTACAGCT
CCATCAGCTCAGCTCGAAAAAGAGCTCCAGGCCCTGGAGAAGGAAAATGCACAGCT
GGAATGGGAGTTGCAAGCACTGGAAAAGGAACTGGCTCAG
SEQ ID NO: 26: mRNA sequence of anti-mouse CD19 CAR
UACCGUUCAGGAGACUGAUCCAAGGAAAGGGAAUUAGAAAACGAGAACCC
UCUCAGUUAAUAAGAACCCUCACCGCUCCGACUAUAGGUCUACUGGGUCUCGGGA
CGAAGUAACAGUUGAAGUGAACCGCUCUGGCAAUGGUAUGUUACAGUUCGGAGG
CUUCUAUAAAUAUCGCCUGACCGUACCAUAGUCGUCUUCGGGCCAUUCUCAGGUG
UUGAAGAGUAAAUGCCGCGGUCACUAGAAGUCCUGCCCCAGGGGUCAUCCAAGA
GACCUAGUCCUAGUCCAUGUGUCAUAUCGGAAUUCUAGUGUUCAUACGUCUGUC
UCCUGCUCCCCCAUAUGAAAACGGUCGUCCCAGAAUGUAUGGGAGCGUGAAAGCC
ACCUCCAUGUUUCGACCUCGACUUUCCCCCUCCACCUUCACCUCCGCCCCCGUCAC
CACCCCCUCCAUCACUCCACGUCAACGUCGUUUCACCCCGUCUUGAACAUGCGGG
GCCGUGGUCGCAAUUUGAAUCAACGUUCCACUCACCCCUAUGGUAUUGAAAAAU
AAUAUACGUGAAGCAUUUUGUUUCUGGUCCAGUCCCCGAACUUACCUAGCCGGCC
UAUCUGGGGCUUCUACUUAGUUGGUUCAUAAGUCUUUUUAAAUUUUUGUUCCGA
UGUGACUGUCGGCUAUGAUCGAGUUUGUGUCGAAUGGACUUCGACAGAUCAAAC
UGAUCACUCCUGUGGCGUUGAAUAAAGACGUAGAUGCCACCUAUGAUAAAACUG
AUGACCCCGGUUCCCCAUUACCAAUGUCAUUCAAGAUGUUGUUGUUUCGGACAU
GACUCCUGUGGAUCAGGACAAGUGGGGUGACCCUGGAGGGUCGGAGUUUCUGGA
CUUCUGACAGCUGGGUCUCCCUCACACUUUCCAUGUCCCAACCUAAAACGUACAC
UAUAAAUGUAGACCCGAGGUGACCGGCCUUAUACGCAUCGUGAGGAGGAGUCGG
AGUAGUAUUGUAACUAGACAAUAGUGUCUAGUUCUUUAUCAGCCUCCUUAGCCG
AAGAAGUUUCGCUGAUGUACUUGUACUGAGGUUCUUCUGGGCCGGAGUGGGCUU
UUGGGAUGGUCGGAAUACGGGGUCGGUCUCUAAAGCGUCGAAUGGCGGGGUCAG
CCAGGCGGCUCUGUCGGCGGUUGGAAGUCCUAGGGUUGGUCGAGAUAUUGCUCA
ACUUAAACCCGGCUUCCCUCCUUAUACUACACGAGCUUUUCUUUGCCCGUGCUCU
AGGGCUUUACCCACCGUUUGUUGUUGCCUCUGCCUUAGGAGUCCUUCCGCAAAUA
UUGCGAGAAGUCUUUCUGUUUUACCGUCUCCGAAUAUCACUCUAACCAUGAUUCC
CCCUUGCCGCCGCUCCCUUUCCAGUGCUGCCGGACAUGGUCCCUGAGAGGUGUCG
UUGUUUCCUAUGUAUGCUACGAGACGUAUACGUCUGGGACCGA
SEQ ID NO: 27: mRNA sequence of anti-human CD19 CAR
UACCGGAAUGGUCACUGGCGGAACGAGGACGGCGACCGGAACGACGAGGU
GCGGCGGUCCGGCCUGUAGGUCUACUGUGUCUGAUGUAGGAGGGACAGACGGAG
AGACCCUCUGUCUCAGUGGUAGUCAACGUCCCGUUCAGUCCUGUAAUCAUUUAUA
AAUUUAACCAUAGUCGUCUUUGGUCUACCUUGACAAUUUGAGGACUAGAUGGUA
UGUAGUUCUAAUGUGAGUCCUCAGGGUAGUUCCAAGUCACCGUCACCCAGACCUU
GUCUAAUAAGAGAGUGGUAAUCGUUGGACCUCGUUCUUCUAUAACGGUGAAUGA
AAACGGUUGUCCCAUUAUGCGAAGGCAUGUGCAAGCCUCCCCCCUGGUUCGACCU
CUAGUGUCCACCGCCACCGAGCCCGCCACCACCCAGCCCACCGCCGCCUAGACUCC
ACUUUGACGUCCUCAGUCCUGGACCGGACCACCGCGGGAGUGUCUCGGACAGGCA
GUGUACGUGACAGAGUCCCCAGAGUAAUGGGCUGAUACCACAUUCGACCUAAGC
GGUCGGAGGUGCUUUCCCAGACCUCACCGACCCUCAUUAUACCCCAUCACUUUGG
UGUAUGAUAUUAAGUCGAGAGUUUAGGUCUGACUGGUAGUAGUUCCUGUUGAGG
UUCUCGGUUCAAAAGAAUUUUUACUUGUCAGACGUUUGACUACUGUGUCGGUAA
AUGAUGACACGGUUUGUAAUAAUGAUGCCACCAUCGAUACGAUACCUGAUGACC
CCGGUUCCUUGGAGUCAGUGGCAGAGGAGUUGGUGCUGCGGUCGCGGCGCUGGU
GGUUGUGGCCGCGGGUGGUAGCGCAGCGUCGGGGACAGGGACGCGGGUCUCCGC
ACGGCCGGUCGCCGCCCCCCGCGUCACGUGUGCUCCCCCGACCUGAAGCGGACAC
UAUAGAUGUAGACCCGCGGGAACCGGCCCUGAACACCCCAGGAAGAGGACAGUGA
CCAAUAGUGGGAAAUGACGUUUGCCCCGUCUUUCUUUGAGGACAUAUAUAAGUU
UGUUGGUAAAUACUCUGGUCAUGUUUGAUGAGUUCUCCUUCUACCGACAUCGAC
GGCUAAAGGUCUUCUUCUUCUUCCUCCUACACUUGACUCUCACUUCAAGUCGUCC
UCGCGUCUGCGGGGGCGCAUGUUCGUCCCGGUCUUGGUCGAGAUAUUGCUCGAG
UUAGAUCCUGCUUCUCUCCUCAUGCUACAAAACCUGUUCUCUGCACCGGCCCUGG
GACUCUACCCCCCUUUCGGCUCUUCCUUCUUGGGAGUCCUUCCGGACAUGUUACU
UGACGUCUUUCUAUUCUACCGCCUCCGGAUGUCACUCUAACCCUACUUUCCGCUC
GCGGCCUCCCCGUUCCCCGUGCUACCGGAAAUGGUCCCAGAGUCAUGUCGGUGGU
UCCUGUGGAUGCUGCGGGAAGUGUACGUCCGGGACGGGGGAGCG
SEQ ID NO: 29: RNA sequence of IL15 single guide RNA #4
CCUGCUGCAGAGUCUGGAAGG
SEQ ID NO: 30; RNA sequence of IL2 single guide RNA #1
GGUAAUGCUUUCUGCCACAC
SEQ ID NO: 31, mRNA sequence of anti-human BCMA CAR
ATGCTGCTCCTCGTAACGAGTCTCCTTCTTTGCGAGCTCCCGCACCCTGCATTTTTGC
TCATACCCCAGATTCAATTGGTCCAATCCGGACCTGAGCTCAAAAAACCAGGGGAA
ACTGTGAAGATCTCATGCAAAGCTTCAGGATATACATTTACTGATTATAGCATAAAT
TGGGTCAAGCGGGCGCCCGGAAAGGGCCTTAAGTGGATGGGTTGGATAAACACCGA
GACAAGAGAACCTGCGTATGCCTACGATTTTAGGGGGCGCTTTGCATTTTCTTTGGA
AACCTCCGCTTCAACTGCATACCTCCAGATTAACAATTTGAAATATGAAGATACCGC
GACATACTTTTGCGCATTGGATTACTCATATGCGATGGATTACTGGGGTCAGGGTAC
TAGCGTAACTGTTAGCTCTGGGGGCGGTGGTAGCGGCGGTGGGGGGAGCGGCGGAG
GCGGGAGTGACATTGTTTTGACCCAGAGCCCTCCATCACTCGCGATGAGCCTGGGTA
AACGGGCAACGATAAGTTGCCGCGCAAGCGAATCCGTGACTATTCTGGGATCACAC
CTGATACACTGGTATCAACAAAAGCCCGGGCAGCCACCAACACTCTTGATACAATT
GGCCTCCAACGTACAAACCGGTGTACCCGCCAGGTTCTCTGGGAGTGGCTCACGGA
CTGATTTCACGCTTACCATCGACCCTGTAGAAGAAGACGATGTCGCAGTCTATTATT
GCCTGCAAAGCCGCACTATACCGAGGACCTTCGGGGGAGGGACCAAGCTTGAGATC
AAGAACTGGAGTCATCCCCAATTCGAAAAAGGTGGTGGCAGCGGCGGGGGATCTGG
GGGTAACTGGAGCCATCCCCAGTTCGAGAAGGGCGGAGGGGGAAGCGGAGGGGGC
GGATCCAACTGGTCTCACCCTCAATTCGAGAAAGGAGGTGGTTCAGGAGGAGGCTC
AGGCGGCGAATCAAAATATGGCCCACCCTGCCCACCGTGTCCAATGTTTTGGGTTCT
GGTTGTTGTTGGCGGCGTACTTGCCTGCTACTCACTGCTGGTGACTGTTGCCTTTATT
ATTTTCTGGGTCAGGTCCAAAAGGGGGCGCAAGAAACTCCTTTACATTTTCAAGCAG
CCATTTATGCGGCCGGTCCAAACCACTCAAGAGGAAGATGGCTGTTCCTGCAGGTTC
CCTGAAGAGGAAGAGGGAGGGTGTGAGCTCCGCGTTAAATTCAGTAGAAGTGCAGA
TGCTCCCGCATACCAGCAGGGGCAAAATCAATTGTATAACGAGCTTAATCTCGGCA
GACGCGAGGAGTATGACGTATTGGACAAACGACGCGGCCGAGATCCAGAAATGGG
GGGGAAGCCTCGGCGGAAAAATCCTCAAGAAGGATTGTACAATGAATTGCAAAAG
GACAAGATGGCTGAGGCCTATAGTGAGATTGGGATGAAAGGAGAAAGGCGCCGAG
GTAAGGGGCACGACGGTCTGTACCAGGGTTTGAGCACAGCCACGAAGGATACGTAT
GACGCGCTGCATATGCAGGCCTTGCCGCCCAGG
SEQ ID NO: 32, mRNA sequence of mouse IL15 sequence
AUGAAAAUUUUGAAACCAUAUAUGAGGAAUACAUCCAUCUCGUGCUACUUGUG
UUUCCUUCUAAACAGUCACUUUUUAACUGAGGCUGGCAUUCAUGUCUUCAUUU
UGGGCUGUGUCAGUGUAGGUCUCCCUAAAACAGAGGCCAACUGGAUAGAUGUA
AGAUAUGACCUGGAGAAAAUUGAAAGCCUUAUUCAAUCUAUUCAUAUUGACAC
CACUUUAUACACUGACAGUGACUUUCAUCCCAGUUGCAAAGUUACUGCAAUGA
ACUGCUUUCUCCUGGAAUUGCAGGUUAUUUUACAUGAGUACAGUAACAUGACU
CUUAAUGAAACAGUAAGAAACGUGCUCUACCUUGCAAACAGCACUCUGUCUUC
UAACAAGAAUGUAGCAGAAUCUGGCUGCAAGGAAUGUGAGGAGCUGGAGGAGA
AAACCUUCACAGAGUUUUUGCAAAGCUUUAUACGCAUUGUCCAAAUGUUCAUC
AACACGUCCUGA
SEQ ID NO: 33, mRNA sequence of human IL15 superagonist
AUGGCACCACGCAGAGCCAGAGGUUGCCGGACUCUCGGACUGCCCGCACUGCUG
CUCCUCCUGCUUCUUCGGCCGCCUGCCACUAGAGGGGACUACAAGGACGACGAU
GACAAGAUCGAAGGGAGGAUUACGUGUCCUCCCCCGAUGUCCGUGGAACACGC
GGACAUCUGGGUCAAGUCCUAUUCCUUGUACUCCCGCGAGCGGUACAUUUGCA
ACUCCGGCUUUAAGCGCAAAGCUGGCACCAGCUCCCUCACUGAAUGCGUGCUGA
ACAAGGCCACUAAUGUGGCCCAUUGGACCACCCCCUCGCUGAAGUGCAUCCGGG
ACCCUGCCUUGGUCCACCAACGCCCUGCACCUCCAUCCGGAGGAUCAGGCGGAG
GAGGUUCGGGUGGUGGUUCCGGUGGAGGAGGGAGCCUCCAGAACUGGGUGAAC
GUGAUCAGCGACCUUAAGAAAAUCGAGGAUCUGAUUCAGUCAAUGCACAUCGA
CGCGACCCUCUACACCGAAAGCGACGUCCACCCGAGCUGCAAGGUCACCGCCAU
GAAGUGCUUCCUGCUGGAACUCCAAGUCAUUUCGCUGGAGAGCGGCGAUGCUU
CAAUCCACGACACUGUGGAAAACCUGAUCAUUCUGGCAAACAACUCCCUCUCUU
CGAAUGGGAACGUGACCGAGUCCGGCUGCAAGGAGUGCGAGGAGCUGGAGGAA
AAGAACAUCAAAGAGUUCCUGCAGUCCUUCGUCCACAUCGUGCAGAUGUUCAU
CAACACCUCG

1. A composition comprising:
a nanoparticle;
a major histocompatibility complex (MHC) molecule; and
a peptide.

2. The composition of claim 1, wherein the MHC molecule comprises an MHC class 1 molecule or an MHC class II molecule.

3. The composition of claim 1, wherein the MHC molecule comprises HLA-A*01:01, HLA-A*02:01, HLA-A*03:01, HLA-A*07:02, HLA-A*11:01, or HLA-A*24:02.

4. The composition of claim 2, wherein the MHC class I molecule comprises a heavy chain that comprises a C-terminal cysteine.

5. The composition of claim 4, wherein the heavy chain comprises a sequence at least 60% identical to one of SEQ ID NOS: 18-20.

6. The composition of claim 1, wherein the peptide binds to the MHC molecule.

7. (canceled)

8. The composition of claim 1, wherein the peptide is a viral protein.

9. The composition of claim 8, wherein the viral protein is an influenza virus protein, a human papillomavirus protein, a human immunodeficiency virus protein, a lymphocytic choriomeningitis virus protein, a cytomegalovirus protein, an Epstein-Barr virus protein, or a SARS-CoV-2 protein.

10. The composition of claim 1, wherein the peptide is expressed on a cancer cell.

11. The composition of claim 1, wherein the peptide comprises a sequence at least 80% identical to one of SEQ ID NOs: 1-9.

12. The composition of claim 1, wherein the nanoparticle is a lipid nanoparticle.

13. The composition of claim 12, wherein the lipid nanoparticle comprises MC3, phosphatidylcholine (1,2-distearoyl-sn-glycero-3-phosphocholin (DSPC), cholesterol, distearoyl glycerol-polyethylene glycol (DMG-PEG), or 1,2-Distearoyl-sn-glycero-3-phosphorylethanolamine (DSPE)-PEG.

14. The composition of claim 1, wherein the MHC molecule and the peptide are located on the surface of the nanoparticle.

15. The composition of claim 1, wherein the density of the MHC molecule on the nanoparticle is about 0.6-0.7 microgram pMHC per microgram lipids.

16. The composition of claim 1, wherein the MHC molecule is conjugated to a linker at the C-terminus.

17. The composition of claim 16, wherein the MHC molecule is linked to the nanoparticle through the linker.

18. The composition of claim 16, wherein the linker comprises 1,2-Distearoyl-sn-glycero-3-phosphorylethanolamine (DSPE), polyethylene glycol (PEG), or maleimide.

19. The composition of claim 1, wherein the nanoparticle further comprises a nucleic acid.

20. (canceled)

21. (canceled)

22. The composition of claim 19, wherein the nucleic acid comprises a sequence of one of SEQ ID NOS: 26-27 and 31-33.

23. A high throughput method of creating a composition that comprises a nanoparticle, a major histocompatibility complex (MHC) molecule, and a peptide, said method comprising:
attaching the MHC molecule to a sacrificial peptide;
contacting the MHC molecule with a lipid micelle to create a first MHC micelle;
contacting the first MHC micelle with the peptide;
applying a UV light to the first MHC micelle and the peptide to create a second MHC micelle; and
creating the composition by contacting the second MHC micelle with the nanoparticle.

24.-44. (canceled)


SEQUENCE LISTING (33 sequences)

SEQ ID NO: 1 - protein, 9 aa, Artificial Sequence
  Synthetic Construct
KAVYNFATM

SEQ ID NO: 2 - protein, 9 aa, Artificial Sequence
  Synthetic Construct
ASNENMETM

SEQ ID NO: 3 - protein, 10 aa, Artificial Sequence
  Synthetic Construct
SSLENFRAYV

SEQ ID NO: 4 - protein, 9 aa, Artificial Sequence
  Synthetic Construct
SSYRRPVGI

SEQ ID NO: 5 - protein, 9 aa, Artificial Sequence
  Synthetic Construct
LSLRNPILV

SEQ ID NO: 6 - protein, 9 aa, Artificial Sequence
  Synthetic Construct
RLIQNSLTI

SEQ ID NO: 7 - protein, 9 aa, Artificial Sequence
  Synthetic Construct
KVPRNQDWL

SEQ ID NO: 8 - protein, 8 aa, Artificial Sequence
  Synthetic Construct
SIINFEKL

SEQ ID NO: 9 - protein, 8 aa, Artificial Sequence
  Synthetic Construct
RTFSFQLI

SEQ ID NO: 10 - protein, 9 aa, Artificial Sequence
  Synthetic Construct
  MISC_FEATURE (6)..(6): photocleavable amino acid
ASNENXETM

SEQ ID NO: 11 - protein, 9 aa, Artificial Sequence
  Synthetic Construct
  MISC_FEATURE (7)..(7): photocleavable amino acid
FAPGNYXAL

SEQ ID NO: 12 - protein, 9 aa, Artificial Sequence
  Synthetic Construct
  misc_feature (8)..(8): Xaa can be any naturally occurring amino acid
  MISC_FEATURE (9)..(9): photocleavable amino acid
YPNVNIHXF

SEQ ID NO: 13 - protein, 9 aa, Artificial Sequence
  Synthetic Construct
  MISC_FEATURE (6)..(6): photocleavable amino acid
STAPGXLEY

SEQ ID NO: 14 - protein, 9 aa, Artificial Sequence
  Synthetic Construct
  MISC_FEATURE (8)..(8): photocleavable amino acid
KILGFVFXV

SEQ ID NO: 15 - protein, 9 aa, Artificial Sequence
  Synthetic Construct
  MISC_FEATURE (5)..(5): photocleavable amino acid
RIYRXGATR

SEQ ID NO: 16 - protein, 9 aa, Artificial Sequence
  Synthetic Construct
  MISC_FEATURE (5)..(5): photocleavable amino acid
AARGXTLAM

SEQ ID NO: 17 - protein, 9 aa, Artificial Sequence
  Synthetic Construct
  MISC_FEATURE (5)..(5): photocleavable amino acid
RVFAXSFIK

SEQ ID NO: 18 - DNA, 873 bp, Artificial Sequence
  Synthetic Construct
atgggcccacactcgatgcggtatttcgagaccgccgtgtcccggcccggcctcgaggagccccggtacatctctgtcggctatgtggacaacaaggagttcgtgcgcttcgacagcgacgcggagaatccgagatatgagccgcgggcgccgtggatggagcaggaggggccggagtattgggagcgggaaacacagaaagccaagggccaagagcagtggttccgagtgagcctgaggaacctgctcggctactacaaccagagcgcgggcggctctcacacactccagcagatgtctggctgtgacttggggtcggactggcgcctcctccgcgggtacctgcagttcgcctatgaaggccgcgattacatcgccctgaacgaagacctgaaaacgtggacggcggcggacatggcggcgcagatcacccgacgcaagtgggagcagagtggtgctgcagagcattacaaggcctacctggagggcgagtgcgtggagtggctccacagatacctgaagaacgggaacgcgacgctgctgcgcacagattccccaaaggcacatgtgacccatcaccccagatctaaaggtgaagtcaccctgaggtgctgggccctgggcttctaccctgctgacatcaccctgacctggcagttgaatggggaggagctgacccagaaaatggagcttgtggagaccaggcctgcaggggatggaaccttccagaagtgggcatctgtggtggtgcctcttgggaaggagcagaattacacatgccgtgtgtaccatgaggggctgcctgagcccctcaccctgagatgggagcctcctccatccactggcagcggtggttccggtggttccgcgtgt

SEQ ID NO: 19 - DNA, 873 bp, Artificial Sequence
  Synthetic Construct
atgggtccacactctctgcgctatttcgttacggctgttagccgtccgggtctgggtgagccgcgctacatggaagtcggttacgtcgacgacaccgaattcgtgcgtttcgacagcgacgcggagaacccgcgttatgagccgcgtgcgcgttggatggagcaggaaggtccggagtactgggagcgtgaaacgcaaaaggcgaagggcaatgaacagagctttcgtgttgatctgcgcactctgctgggttactacaaccagagcaaaggtggcagccataccattcaggtgattagcggttgtgaagtcggctctgatggccgcctgttgcgcggttatcagcaatatgcatacgacggttgcgactacattgcgctgaatgaagatctgaaaacgtggactgcggcggacatggccgcactgattaccaaacacaagtgggagcaagcgggcgaagccgagcgcctgcgtgcgtatctggaaggcacctgtgtggaatggctgcgccgctatctgaagaatggcaatgccacgttgctgcgtacggattccccgaaagcgcacgtgacgcaccatagccgtcctgaggataaagttaccctgcgttgctgggcactgggcttttacccggcagatatcaccttgacgtggcaactgaatggtgaagagctgattcaggatatggaactggtggagactcgtccggctggcgacggtaccttccagaaatgggcatcggttgtcgtccctctgggtaaagagcaatactatacctgccacgtttaccaccaaggtctgccggagccgctgaccttgcgttgggagccaccgccgagcaccggcagcggtggtagcggcggttccgcgtgt

SEQ ID NO: 20 - DNA, 840 bp, Artificial Sequence
  Synthetic Construct
atgggttctcattctatgagatatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacggacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatgggagccggctagctgc

SEQ ID NO: 21 - DNA, 300 bp, Artificial Sequence
  Synthetic Construct
atccagcgtaccccgaagattcaggtgtactcacgccatccagctgagaacggcaaaagtaactttctgaattgctatgtctcgggcttccacccgtccgatatcgaagtggaccttctgaaaaacggtgaacggatcgaaaaagtcgagcattctgacttatcattcagcaaagattggtctttttacttgttatattataccgaatttaccccgacagaaaaagatgagtatgcatgccgtgtcaaccacgttacgctgtcccagccgaaaatcgtgaaatgggatcgtgatatgtaa

SEQ ID NO: 22 - DNA, 786 bp, Artificial Sequence
  Synthetic Construct
atgccgcgcagcagagctctgattctgggggtcctcgccctgaacaccatgctcagcctctgcggaggtgaagacgacattgaggccgaccacgtaggcttctatggtacaactgtttatcagtctcctggagacattggccagtacacacatgaatttgatggtgatgagttgttctatgtggacttggataagaagaaaactgtctggaggcttcctgagtttggccaattgatactctttgagccccaaggtggactgcaaaacatagctgcagaaaaacacaacttgggaatcttgactaagaggtcaaatttcaccccagctaccaatgaggctcctcaagcgactgtgttccccaagtcccctgtgctgctgggtcagcccaacacccttatctgctttgtggacaacatcttcccacctgtgatcaacatcacatggctcagaaatagcaagtcagtcacagacggcgtttatgagaccagcttcctcgtcaaccgtgaccattccttccacaagctgtcttatctcaccttcatcccttctgatgatgacatttatgactgcaaggtggagcactggggcctggaggagccggttctgaaacactgggaacctgagattccagcccccatgtcagagctgacagaggtcgacactacagctccatcagctcagttgaaaaagaaattgcaagcactgaagaaaaagaacgctcagctgaagtggaaacttcaagccctcaagaagaaactcgcccagtccgggagcggatctggctgt

SEQ ID NO: 23 - DNA, 702 bp, Artificial Sequence
  Synthetic Construct
ggagactccgaaaggcatttcgtgcaccagttcaagggcgagtgctacttcaccaacgggacgcagcgcatacggctcgtgaccagatacatctacaaccgggaggagtacctgcgcttcgacagcgacgtgggcgagtaccgcgcggtgaccgagctggggcggcactcagccgagtactacaataagcagtacctggagcgaacgcgggccgagctggacacggcgtgcagacacaactacgaggagacggaggtccccacctccctgcggcggcttgaacagcccaatgtcgccatctccctgtccaggacagaggccctcaaccaccacaacactctggtctgttcggtgacagatttctacccagccaagatcaaagtgcgctggttcaggaatggccaggaggagacagtgggggtatcatccacacagcttattaggaatggggactggaccttccaggtcctggtcatgctggagatgacccctcatcagggagaggtctacacctgccatgtggagcatcccagcctgaagagccccatcactgtggagtggagggcacagtccgagtctgcccggagcaagggatccactacagctccatcagctcagctcgaaaaagagctccaggccctggagaaggaaaatgcacagctggaatgggagttgcaagcactggaaaaggaactggctcag

SEQ ID NO: 24 - DNA, 786 bp, Artificial Sequence
  Synthetic Construct
atgccgcgcagcagagctctgattctgggggtcctcgccctgaccaccatgctcagcctctgtggaggtgaagacgacattgaggccgaccacgtaggcacctatggtataagtgtatatcagtctcctggagacattggccagtacacatttgaatttgatggtgatgagttgttctatgtggacttggataagaaggagactgtctggatgcttcctgagtttggccaattggcaagctttgacccccaaggtggactgcaaaacatagctgtagtaaaacacaacttgggagtcttgactaagaggtcaaattccaccccagctaccaatgaggctcctcaagcgactgtgttccccaagtcccctgtgctgctgggtcagcccaacaccctcatctgctttgtggacaacatcttccctcctgtgatcaacatcacatggctcagaaatagcaagtcagtcgcagacggtgtttatgagaccagcttcttcgtcaaccgtgactattccttccacaagctgtcttatctcaccttcatcccttctgacgatgacatttatgactgcaaggtggaacactggggcctggaggagccggttctgaaacactgggaacctgagattccagcccccatgtcagagctgacagaggtcgacactacagctccatcagctcagttgaaaaagaaattgcaagcactgaagaaaaagaacgctcagctgaagtggaaacttcaagccctcaagaagaaactcgcccagtccgggagcggatctggctgt

SEQ ID NO: 25 - DNA, 708 bp, Artificial Sequence
  Synthetic Construct
ggagactccgaaaggcatttcgtgtaccagttcatgggcgagtgctacttcaccaacgggacgcagcgcatacgatatgtgaccagatacatctacaaccgggaggagtacgtgcgctacgacagcgacgtgggcgagcaccgcgcggtgaccgagctggggcggccagacgccgagtactggaacagccagccggagatcctggagcgaacgcgggccgagctggacacggtgtgcagacacaactacgaggggccggagacccacacctccctgcggcggcttgaacagcccaatgtcgtcatctccctgtccaggacagaggccctcaaccaccacaacactctggtctgctcagtgacagatttctacccagccaagatcaaagtgcgctggttccggaatggccaggaggagacggtgggggtctcatccacacagcttattaggaatggggactggaccttccaggtcctggtcatgctggagatgacccctcggcggggagaggtctacacctgtcacgtggagcatcccagcctgaagagccccatcactgtggagtggagggcacagtctgagtctgcccggagcaagggatccactacagctccatcagctcagctcgaaaaagagctccaggccctggagaaggaaaatgcacagctggaatgggagttgcaagcactggaaaaggaactggctcag

SEQ ID NO: 26 - RNA, 1455 nt, Artificial Sequence
  Synthetic Construct
uaccguucaggagacugauccaaggaaagggaauuagaaaacgagaacccucucaguuaauaagaacccucaccgcuccgacuauaggucuacugggucucgggacgaaguaacaguugaagugaaccgcucuggcaaugguauguuacaguucggaggcuucuauaaauaucgccugaccguaccauagucgucuucgggccauucucagguguugaagaguaaaugccgcggucacuagaaguccugccccaggggucauccaagagaccuaguccuaguccaugugucauaucggaauucuaguguucauacgucugucuccugcucccccauaugaaaacggucgucccagaauguaugggagcgugaaagccaccuccauguuucgaccucgacuuucccccuccaccuucaccuccgcccccgucaccacccccuccaucacuccacgucaacgucguuucaccccgucuugaacaugcggggccguggucgcaauuugaaucaacguuccacucaccccuaugguauugaaaaauaauauacgugaagcauuuuguuucugguccaguccccgaacuuaccuagccggccuaucuggggcuucuacuuaguugguucauaagucuuuuuaaauuuuuguuccgaugugacugucggcuaugaucgaguuugugucgaauggacuucgacagaucaaacugaucacuccuguggcguugaauaaagacguagaugccaccuaugauaaaacugaugaccccgguuccccauuaccaaugucauucaagauguuguuguuucggacaugacuccuguggaucaggacaaguggggugacccuggagggucggaguuucuggacuucugacagcugggucucccucacacuuuccaugucccaaccuaaaacguacacuauaaauguagacccgaggugaccggccuuauacgcaucgugaggaggagucggaguaguauuguaacuagacaauagugucuaguucuuuaucagccuccuuagccgaagaaguuucgcugauguacuuguacugagguucuucugggccggagugggcuuuugggauggucggaauacggggucggucucuaaagcgucgaauggcggggucagccaggcggcucugucggcgguuggaaguccuaggguuggucgagauauugcucaacuuaaacccggcuucccuccuuauacuacacgagcuuuucuuugcccgugcucuagggcuuuacccaccguuuguuguugccucugccuuaggaguccuuccgcaaauauugcgagaagucuuucuguuuuaccgucuccgaauaucacucuaaccaugauucccccuugccgccgcucccuuuccagugcugccggacauggucccugagaggugucguuguuuccuauguaugcuacgagacguauacgucugggaccga

SEQ ID NO: 27 - RNA, 1458 nt, Artificial Sequence
  Synthetic Construct
uaccggaauggucacuggcggaacgaggacggcgaccggaacgacgaggugcggcgguccggccuguaggucuacugugucugauguaggagggacagacggagagacccucugucucagugguagucaacgucccguucaguccuguaaucauuuauaaauuuaaccauagucgucuuuggucuaccuugacaauuugaggacuagaugguauguaguucuaaugugaguccucaggguaguuccaagucaccgucacccagaccuugucuaauaagagagugguaaucguuggaccucguucuucuauaacggugaaugaaaacgguugucccauuaugcgaaggcaugugcaagccuccccccugguucgaccucuaguguccaccgccaccgagcccgccaccacccagcccaccgccgccuagacuccacuuugacguccucaguccuggaccggaccaccgcgggagugucucggacaggcaguguacgugacagaguccccagaguaaugggcugauaccacauucgaccuaagcggucggaggugcuuucccagaccucaccgacccucauuauaccccaucacuuugguguaugauauuaagucgagaguuuaggucugacugguaguaguuccuguugagguucucgguucaaaagaauuuuuacuugucagacguuugacuacugugucgguaaaugaugacacgguuuguaauaaugaugccaccaucgauacgauaccugaugaccccgguuccuuggagucaguggcagaggaguuggugcugcggucgcggcgcuggugguuguggccgcgggugguagcgcagcgucggggacagggacgcgggucuccgcacggccggucgccgccccccgcgucacgugugcucccccgaccugaagcggacacuauagauguagacccgcgggaaccggcccugaacaccccaggaagaggacagugaccaauagugggaaaugacguuugccccgucuuucuuugaggacauauauaaguuuguugguaaauacucuggucauguuugaugaguucuccuucuaccgacaucgacggcuaaaggucuucuucuucuuccuccuacacuugacucucacuucaagucguccucgcgucugcgggggcgcauguucgucccggucuuggucgagauauugcucgaguuagauccugcuucucuccucaugcuacaaaaccuguucucugcaccggcccugggacucuaccccccuuucggcucuuccuucuugggaguccuuccggacauguuacuugacgucuuucuauucuaccgccuccggaugucacucuaacccuacuuuccgcucgcggccuccccguuccccgugcuaccggaaauggucccagagucaugucggugguuccuguggaugcugcgggaaguguacguccgggacgggggagcg

SEQ ID NO: 28 - protein, 9 aa, Artificial Sequence
  Synthetic Construct
  MISC_FEATURE (7)..(7): photocleavable amino acid
FAPGNYXAL

SEQ ID NO: 29 - RNA, 21 nt, Artificial Sequence
  Synthetic Construct
ccugcugcagagucuggaagg

SEQ ID NO: 30 - RNA, 20 nt, Artificial Sequence
  Synthetic Construct
gguaaugcuuucugccacac

SEQ ID NO: 31 - DNA, 1554 bp, Artificial Sequence
  Synthetic Construct
atgctgctcctcgtaacgagtctccttctttgcgagctcccgcaccctgcatttttgctcataccccagattcaattggtccaatccggacctgagctcaaaaaaccaggggaaactgtgaagatctcatgcaaagcttcaggatatacatttactgattatagcataaattgggtcaagcgggcgcccggaaagggccttaagtggatgggttggataaacaccgagacaagagaacctgcgtatgcctacgattttagggggcgctttgcattttctttggaaacctccgcttcaactgcatacctccagattaacaatttgaaatatgaagataccgcgacatacttttgcgcattggattactcatatgcgatggattactggggtcagggtactagcgtaactgttagctctgggggcggtggtagcggcggtggggggagcggcggaggcgggagtgacattgttttgacccagagccctccatcactcgcgatgagcctgggtaaacgggcaacgataagttgccgcgcaagcgaatccgtgactattctgggatcacacctgatacactggtatcaacaaaagcccgggcagccaccaacactcttgatacaattggcctccaacgtacaaaccggtgtacccgccaggttctctgggagtggctcacggactgatttcacgcttaccatcgaccctgtagaagaagacgatgtcgcagtctattattgcctgcaaagccgcactataccgaggaccttcgggggagggaccaagcttgagatcaagaactggagtcatccccaattcgaaaaaggtggtggcagcggcgggggatctgggggtaactggagccatccccagttcgagaagggcggagggggaagcggagggggcggatccaactggtctcaccctcaattcgagaaaggaggtggttcaggaggaggctcaggcggcgaatcaaaatatggcccaccctgcccaccgtgtccaatgttttgggttctggttgttgttggcggcgtacttgcctgctactcactgctggtgactgttgcctttattattttctgggtcaggtccaaaagggggcgcaagaaactcctttacattttcaagcagccatttatgcggccggtccaaaccactcaagaggaagatggctgttcctgcaggttccctgaagaggaagagggagggtgtgagctccgcgttaaattcagtagaagtgcagatgctcccgcataccagcaggggcaaaatcaattgtataacgagcttaatctcggcagacgcgaggagtatgacgtattggacaaacgacgcggccgagatccagaaatgggggggaagcctcggcggaaaaatcctcaagaaggattgtacaatgaattgcaaaaggacaagatggctgaggcctatagtgagattgggatgaaaggagaaaggcgccgaggtaaggggcacgacggtctgtaccagggtttgagcacagccacgaaggatacgtatgacgcgctgcatatgcaggccttgccgcccagg

SEQ ID NO: 32 - RNA, 489 nt, Artificial Sequence
  Synthetic Construct
augaaaauuuugaaaccauauaugaggaauacauccaucucgugcuacuuguguuuccuucuaaacagucacuuuuuaacugaggcuggcauucaugucuucauuuugggcugugucaguguaggucucccuaaaacagaggccaacuggauagauguaagauaugaccuggagaaaauugaaagccuuauucaaucuauucauauugacaccacuuuauacacugacagugacuuucaucccaguugcaaaguuacugcaaugaacugcuuucuccuggaauugcagguuauuuuacaugaguacaguaacaugacucuuaaugaaacaguaagaaacgugcucuaccuugcaaacagcacucugucuucuaacaagaauguagcagaaucuggcugcaaggaaugugaggagcuggaggagaaaaccuucacagaguuuuugcaaagcuuuauacgcauuguccaaauguucaucaacacguccuga

SEQ ID NO: 33 - RNA, 759 nt, Artificial Sequence
  Synthetic Construct
auggcaccacgcagagccagagguugccggacucucggacugcccgcacugcugcuccuccugcuucuucggccgccugccacuagaggggacuacaaggacgacgaugacaagaucgaagggaggauuacguguccucccccgauguccguggaacacgcggacaucugggucaaguccuauuccuuguacucccgcgagcgguacauuugcaacuccggcuuuaagcgcaaagcuggcaccagcucccucacugaaugcgugcugaacaaggccacuaauguggcccauuggaccacccccucgcugaagugcauccgggacccugccuugguccaccaacgcccugcaccuccauccggaggaucaggcggaggagguucggguggugguuccgguggaggagggagccuccagaacugggugaacgugaucagcgaccuuaagaaaaucgaggaucugauucagucaaugcacaucgacgcgacccucuacaccgaaagcgacguccacccgagcugcaaggucaccgccaugaagugcuuccugcuggaacuccaagucauuucgcuggagagcggcgaugcuucaauccacgacacuguggaaaaccugaucauucuggcaaacaacucccucucuucgaaugggaacgugaccgaguccggcugcaaggagugcgaggagcuggaggaaaagaacaucaaagaguuccugcaguccuucguccacaucgugcagauguucaucaacaccucg